(19) United States
(12) Patent Application Publication
(10) Pub. No.: US 2010/0298352 A1
(43) Pub. Date: Nov. 25, 2010

(54) SMALL MOLECULE INHIBITORS OF CANCER STEM CELLS

(75) Inventors: Edward V. Prochownik, Pittsburgh, PA (US); John S. Lazo, Pittsburgh, PA (US)

Correspondence Address: KLARQUIST SPARKMAN, LLP, 121 SW SALMON STREET, SUITE 1600, PORTLAND, OR, 97204, US

(73) Assignee: University of Pittsburgh - Of the Commonwealth System of Higher Education

(21) Appl. No.: 12/775,023

(22) Filed: May 6, 2010

Related U.S. Application Data
(60) Provisional application No. 61/176,414, filed on May 7, 2009.

(51) Int. Cl. A61K 31/52; A61K 31/353; A61P 35/00

(52) U.S. Cl. 514/263.4; 514/456

(57) ABSTRACT

A method of inhibiting a carcinoma in a subject, comprising administering to the subject at least one therapeutic agent that selectively targets carcinoma stem cells. Illustrative carcinoma stem cell-selective therapeutic agents include CGP74514A, rottlerin, and A-77636.

### CROSS REFERENCE TO RELATED APPLICATION

This application claims the benefit of priority of U.S. Provisional Application No. 61/176,414, filed May 7, 2009, which is herein incorporated by reference in its entirety.

### FIELD

Disclosed herein are inhibitors of cancer stem cells.

### BACKGROUND

The concept of cancer stem cells (CSCs) is based upon ideas first formulated in the context of the hierarchical hematopoietic system, where normal stem cells have an unlimited capacity for self-renewal that is gradually lost as they generate multi-potent progenitors and their more differentiated progeny. As applied to cancer, the CSC model posits that not all cancer cells are identical in their ability to initiate the growth of a new tumor. In breast cancer, for example, cell surface markers have been used to identify tumor cell subpopulations with widely differing capacities for tumor initiation. Thus, cells with high surface expression of the epithelial marker CD44 (CD44hi) and low-absent expression of CD24 (CD24lo) possess robust tumor initiating activity relative to the remaining population. Recent work has provided evidence that tumors with high levels of CSC markers identify sub-groups of patients who are particularly prone to therapy failure and relapse. The concept of CSCs has been extended beyond breast cancer to other malignancies, including cancer of the prostate, brain (gliomas), colon, and pancreas.

In most cases examined to date, CSCs comprises<1% of tumor cells, with the remainder comprising the more differentiated “transient amplifying cell” (TAC) population. However, the true frequency of CSCs remain somewhat fluid. The CSC hypothesis suggests that, because the bulk of tumor cells have low tumor initiating capacity, their eradication will result in substantial tumor shrinkage but will not be curative unless CSCs are eliminated concurrently. This has led to the proposal that relapses arise as a consequence of inherent differential therapeutic sensitivities of the CSC and TAC compartments. Unfortunately, the paucity of CSCs and their tendency to differentiate into TACs has prevented a full-scale testing of this hypothesis. Further, because CSCs differentiate shortly after being isolated, sufficient numbers of CSCs have not been able to be produced via cell culture.

Oct3/4 is a germline-specific transcription factor, also known as OTF3 or POU5F1) recently reported to play an important role in germ cell specification. Expression of human Oct3/4 is driven by an approximately 4 kb promoter element (GenBank Ace. No. DQ249177.1; FIG. 9). The Oct3/4 promoter contains three virtual open reading frames (ORFs), all oriented in a 3′→5′ direction relative to the sequence depicted in FIG. 9. ORF1 (ca. 1.1-1.6 kb from the 5′ end) could potentially encode a 162 amino acid protein. ORF2 (ca. 2.4-2.8 kb from the 5′ end) could encode a 138 amino acid protein, and ORF3 (ca. 2.7-3.3 kb from the 5′ end) could encode a 201 amino acid protein.

### SUMMARY

Disclosed herein are compounds that selectively inhibit cancer stem cells, and methods for inhibiting cancer that include administering such compounds to a subject.

One method disclosed herein is a method of inhibiting a carcinoma in a subject, comprising administering to the subject at least one therapeutic agent that selectively targets carcinoma stem cells.

According to another embodiment, there is disclosed a method of selectively targeting carcinoma stem cells, comprising administering to a population of carcinoma cells comprising carcinoma stem cells as well as carcinoma cells that are not stem cells at least one anti-cancer agent selected from:

(a) a purine compound, or a pharmaceutically acceptable salt or ester thereof, having a structure represented by

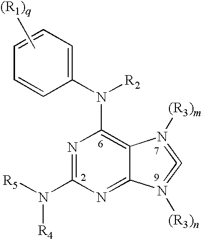

in which q is 1-5,

R₁ is halogen, lower alkyl, hydroxyl or lower alkanoyloxy; lower alkoxy which is unsubstituted or substituted by hydroxyl, lower alkoxy or carboxyl; a radical of the formula —O(—CH₂—CH₂—O)ₜ—R₆, in which t is 2-5 and R₆ is hydrogen or lower alkyl; carboxyl, lower alkoxycarbonyl, piperazin-1-yl-carbonyl or carbamoyl; N-lower alkyl-carbamoyl, which is unsubstituted or substituted by hydroxyl or amino in the lower alkyl moiety; N,N-di-lower alkyl-carbamoyl, cyano, nitro, amino, lower alkanoyl amino, lower alkylamino, N,N-di-lower alkylamino, aminosulfonyl or trifluoromethyl, where, if more than one radical R is present in the molecule, these can be identical to or different from one another,

R₂ is hydrogen, carbamoyl or N-lower alkyl-carbamoyl, m and n are each 0 or 1, where m is 0 if n is 1 and m is 1 if n is 0,

R₃ is lower alkyl or phenyl which are unsubstituted or in each case substituted by hydroxyl, lower alkoxy, amino, lower alkylamino or N,N-di-lower alkyl amino, and

i) R₄ is hydrogen, amino, phenylamino, lower alkylamino, hydroxyl, phenoxy, lower alkoxy, acyl having 1-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms and R₅ is amino, phenylamino, lower alkylamino, hydroxyl, phenoxy, lower alkoxy, acyl having 2-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms, or

ii) R₄ and R₅ together are a substituted or unsubstituted alkylene or alkenylene radical having in each case not more than 15 C atoms, in which 1-3 C atoms can be replaced by oxygen, sulfur or nitrogen, and their salts;

(b) a phenolic compound, or a pharmaceutically acceptable salt or ester thereof, having a structure represented by

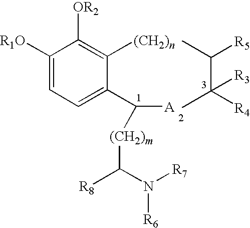

wherein A is O, C, CH, or CH₂;

R₁ and R₂ are independently hydrogen or a leaving group or a protecting group;

m and n are independently selected from zero or 1;

R₃ is H, alkyl, alkenyl, aryl, cycloalkyl, or taken together with R₄ can form a spirocycloalkyl, with the proviso that when n is zero R₃ is not H;

R₄ is H or alkyl, or taken together with R₃ can form a spirocycloalkyl;

R₅ is H or alkyl, or when n is zero, R₅ can be taken together with R₃ to form a fused cycloalkyl;

R₆ is H, alkyl, or taken together with R₈ can form an N containing heterocycle;

R₇ is H, alkyl, alkenyl, cycloalkyl, arylalkyl, or taken together with A when A is C and when m=0 and n=0, can form a fused N containing heterocycle, or taken together with R₈ can form an N containing heterocycle; or R₆ and R₇ together can form an N containing heterocycle with the proviso that when R₆ is alkyl R₇ cannot be arylalkyl;

R₈ is H, alkyl, taken together with R₆ or R₇ to form an N containing heterocycle, or taken together with the catechol ring can form a fused ring; or

(c) a rottlerin compound, or a pharmaceutically acceptable salt or ester thereof, having a structure represented by

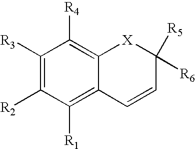

wherein X is selected from the group consisting of CH₂, O, N, and S; R₁ and R₃ are selected from the group consisting of H, OH, NH and SH; R₂ is selected from the group consisting of ethanone, acetyl, alkenyl, aryl and alkyl; R₄ is CO—[CHCH]ₙ-Ph, CN—[CHCH]ₙ-Ph, or COOZ, wherein Z is selected from the group consisting of alkenyl, aryl, and alkyl; R₅ and R₆ are selected from the group consisting of H, OH, NH, SH, alkenyl, aryl, and alkyl; and n is 0 to 5.

A further embodiment disclosed herein is a method of treating a chemotherapeutic-resistant carcinoma, comprising administering to a subject having a chemotherapeutic-resistant carcinoma at least one therapeutic agent that selectively targets carcinoma stem cells.

Another embodiment is a method of inhibiting breast cancer in a subject, comprising administering to the subject a therapeutically effective amount of at least one therapeutic agent that selectively targets breast cancer stem cells, the at least one therapeutic agent being selected from:

(a) a purine compound, or a pharmaceutically acceptable salt or ester thereof, as described above;

(b) a phenolic compound, or a pharmaceutically acceptable salt or ester thereof, as described above; or

(c) a rottlerin compound, or a pharmaceutically acceptable salt or ester thereof, as described above.

Also disclosed herein is a pharmaceutical composition useful for selectively targeting carcinoma stem cells, comprising a therapeutically effective amount of at least one therapeutic agent that selectively targets carcinoma stem cells, the at least one therapeutic agent being selected from:

(a) a purine compound, or a pharmaceutically acceptable salt or ester thereof, as described above;

(b) a phenolic compound, or a pharmaceutically acceptable salt or ester thereof, as described above; or

(c) a rottlerin compound, or a pharmaceutically acceptable salt or ester thereof, as described above; and at least one carrier or adjuvant.

The foregoing will become more apparent from the following detailed description, which proceeds with reference to the accompanying figures.

### BRIEF DESCRIPTION OF THE DRAWINGS

FIG. 1A-B. Distinct cloning behaviors of MCF7 tumor cell subsets. (A). MCF7 cells were stained with monoclonal antibodies (mAbs) directed against human CD44 and CD24 (Becton-Dickinson). The anti-CD44 mAb was tagged with Allophycocyanin (APC) and the anti-CD24 mAb was tagged with Phycoerythrin (PE). Depending on the experiment, CSCs (CD44hi/CD24lo) comprised ca. 0.5-1.5% of the entire tumor cell population. (B) Single cell seeding into 96 well plates was accomplished with a DAKO MoFlo Fluorescence-activated cell sorter. Three plates of CD44hi/CD24lo-enriched MCF7 cells and 3 plates of CD44hi/CD24lo-depleted cells were seeded and allowed to grow into macroscopically identifiable colonies. The cumulative number of expandable clones was then noted as soon as they became visible and is plotted as a function of the time after seeding.

FIG. 2. Evaluation of the surface marker expression in separate CSC and TAC populations of MCF7 cells. Individual CSC(CD44hi/CD24lo) and non-CSC(CD44hi/CD24lo-depleted or TAC) single cell clones from FIG. 1B were expanded and assessed for the expression of CD44 and CD24. Over 15 clones were examined from each group with 3 representative clones from each group being shown here. Note that clones initially derived from CSCs (CD44hi/CD24lo) rapidly reverted so as to now more closely resemble TACs or the uncloned MCF7 cells. The center panel shows the profile of the uncloned, starting MCF7 population, which is similar to that shown in FIG. 1A. Note that in all cases, clones were examined for CD44 and CD24 expression within 3-4 weeks of their initial seeding as single cells.

FIG. 3A-C. Characterization of Oct3/4-GFP+MCF7-derived CSCs. (A). MCF7 cells were transfected by electroporation with a linearized Oct3/4-GFP vector. A separate cassette on the plasmid imparted G-418 resistance under the control of a promiscuous SV40 promoter. The photos show separate phase contrast and fluorescence micrographs of the same field demonstrating the expected low percentage of GFP+ cells among the stable, G418-resistant transfectants. (B). GFP+ cells from the above population were purified by cell sorting, expanded for several days and examined for CD44 and CD24 expression. Note that these cells were comprised of a highly enriched CSC-like CD44hi/CD24lo population. These cells have remained 100% GFP+ for >20 weeks. (C). In the reciprocal experiment, the CD44hi/CD24lo population was isolated from the bulk culture in (A) and then examined for the expression of GFP. Note that virtually all cells expressed GFP (shaded area), a finding confirmed by visual inspection. These cells have all remained GFP+for >15 weeks. As a negative control, non-transfected MCF7 cells were examined in parallel (open area *).

FIG. 4A-B. Morphological differences between GFP+CD44hi/CD24lo CSCs and the bulk of MCF7 cells. (A). Standard hematoxylin & eosin staining of MCF7 cells and Oct3/4-GFP+MCF7 cells. Cells were plated onto glass coverslips, allowed to attach and grow for two days and then fixed under standard conditions. Light microscopic photos were taken under 60× magnification. Note the relative homogeneity of each population with Oct3/4-GFP+MCF7 cells being smaller and rounder. (B). Size differences between GFP+CD44hi/CD24lo CSCs (dashed curve) and the bulk of MCF7 cells (ribbon curve). Cell diameters for each of the two populations were determined on a Beckman-Coulter Vi-Cell Cell Viability Analyzer. >95% of each population was deemed to be viable and only viable cells were included in the analysis. At least 3000 cells from each group were evaluated. Numbers above the curves are the mean diameter of each population.

FIG. 5A-C. The Oct3/4-GFP+MCF7 cell population retains its CSC-like properties after in vivo tumorigenesis. (A). Nude mice were inoculated with 10⁴ Oct3/4-GFP+MCF7 cells. 3/3 animals developed tumors within 2 weeks. These tumors were allowed to grow for an additional 2 weeks at which time they were excised. Single cell suspensions were then prepared and propagated in G-418-containing medium for an additional 2 weeks. At the end of this time, the surviving cells were viewed by visible and UV microscopy. (B). The cells were also subjected to flow cytometry to determine the fraction of cells that were positive for GFP. Note that virtually all of the cells retained expression of GFP (shaded curve). A GFP-population of MCF7 cells was included as a negative control (solid curve *). (C). The GFP+population was also stained for CD44 and CD24. Note that the vast majority of cells displayed the breast cancer CSC phenotype (CD44hi/CD24lo).

FIG. 6. Differential chemotherapeutic responses of MCF7 and Oct3/4-GFP+MCF7 cells. 10⁵ unfractionated, MCF7 cells (dotted line) or Oct3/4GFP+MCF7 cells (broken line) were seeded into 6 well plates and allowed to achieve ca. 30-50% confluence. The indicated concentrations of adriamycin, etoposide, 5-fluorouracil, cis-platinum, methotrexate, and taxol were then added (day 0). The total number of viable adherent cells was then determined at the indicated times by trypan blue exclusion. The results show the average for triplicate plates+/−the SE. Note that in most cases Oct4/4-GFP+MCF7 actually grew in the presence of all agents. These experiments were repeated at least two additional times with similar outcomes.

FIG. 7A-C. Fluorescence-based identification of unfractionated MCF7 cells and Oct3/-GFP+MCF7 CSCs. (A). Unfractionated, non-CSC-MCF7 cells were infected with a lentiviral vector encoding DsRED (Clontech) under the control of the CMV immediate early promoter. They were then mixed with an equivalent number of Oct3/4-GFP+MCF7 cells and photographed with a fluorescence microscope under sequential imaging conditions that captured the individual CFP and DsRED signals. The combined image is shown here.

(B). Fluorescence intensities of DsRED+MCF7 cells and Oct3/4-GFP+MCF7 cells are directly proportional to cell number. The indicated numbers of cells were seeded into individual wells of a 96 well plate and allowed to attach overnight. The following day, fluorescence intensities were determined on quadruplicate samples using a fluorescence plate reader (Molecular Devices, SpectraMax M2). Excitation/Emission parameters were 485/538 for GFP and 584/612 for DsRED. The values shown represent the mean fluorescence intensities obtained after background subtraction (<2%). Maximal values, obtained with the highest cell number, were arbitrarily set at 1. (C). DsRED+MCF7 cells and Oct3/4-GFP+MCF7 cells were mixed at the indicated ratios and seeded into 96 well plates. The following day, fluorescence intensities were quantified as described in (B). The red bar indicates the relative fluorescence intensity of the DsRED+ population that was plated in the set of wells labeled 1:1. This number was held constant while the number of GFP+ cells (green bars) was reduced by the amount indicated.

FIG. 8. Oct3/4 promoter deletion clones that will be used to identify the minimally active region. Schematic diagram of the 3971 by “full-length” (FL) human Oct3/4 promoter. The deletions were produced using the corresponding restriction enzyme cleave sites shown in FIG. 9. In particular, the Oct3/4 promoter sequence of construct B is the NdeI-EcoRI fragment, the Oct3/4 promoter sequence of construct C is the AccI-BamHI fragment, the Oct3/4 promoter sequence of construct D is the SacI-BamHI fragment, the Oct3/4 promoter sequence of construct E is the SalI-BamHI fragment, and the Oct3/4 promoter sequence of construct F is the NdeI-SalI fragment.

FIG. 9. A 3990-nucleotide human Oct3/4 promoter sequence (GenBank Acc. No. DQ249177.1; SEQ ID NO:1). The 3′ end of the sequence has been abutted to GFP in the construct used for MCF7 electroporations. Several pertinent restriction sites that were used for the construction of deletion mutants shown in the accompanying figure are depicted in italics.

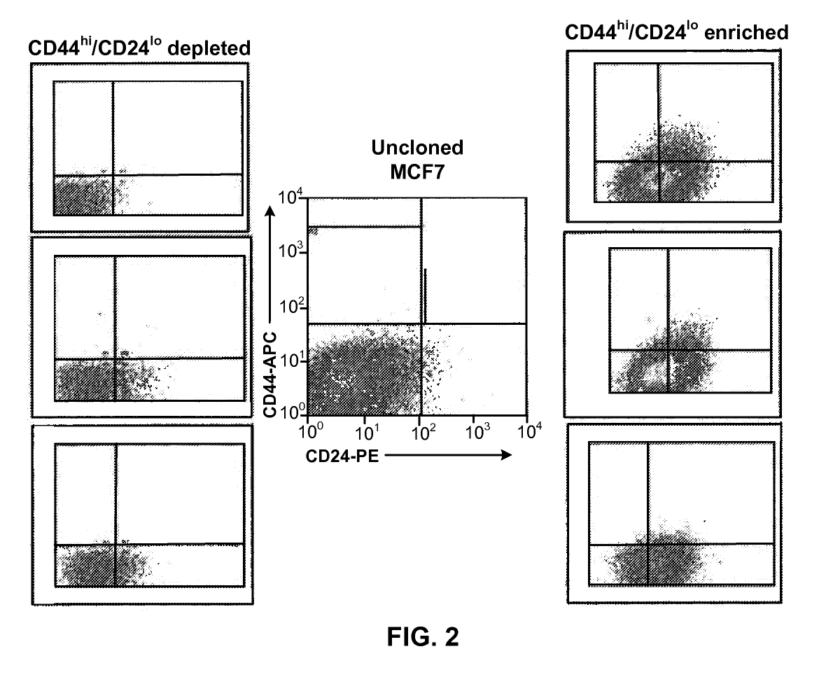

FIG. 10. Nucleotide sequence of the murine Oct3/4 promoter, residues+11 through −1879 from FIG. 2 of Okazawa et al., 1991, EMBO J. 10:2997-3005 (SEQ ID NO:8).

FIG. 11. Several graphs showing selective inhibition of Oct3/4-GFP+MCF cells with several different small molecules.

### DETAILED DESCRIPTION

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

As used herein, the singular terms “a,” “an,” and “the” include plural referents unless context clearly indicates otherwise. Also, as used herein, the term “comprises” means “includes.”

The term “acyl” refers to a group of the formula RC(O)— wherein R is an organic group.

“Administration of and “administering a” compound should be understood to mean providing a compound, a prodrug of a compound, or a pharmaceutical composition as described herein. The compound or composition can be administered by another person to the subject (e.g., intravenously) or it can be self-administered by the subject (e.g., tablets).

The term “aliphatic” is defined as including alkyl, alkenyl, alkynyl, halogenated alkyl and cycloalkyl groups as described above. A “lower aliphatic” group is a branched or unbranched aliphatic group having from 1 to 10 carbon atoms.

“Alkanediyl” or “cycloalkanediyl” refers to a divalent radical of the general formula —CₙH₂ₙ— derived from aliphatic or cycloaliphatic hydrocarbons.

The term “alkenyl” refers to a hydrocarbon group of 2 to 24 carbon atoms and structural formula containing at least one carbon-carbon double bond. A “lower alkenyl” group has 1 to 10 carbon atoms.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, or carboxyl.

The term “alkyl amino” refers to alkyl groups as defined above where at least one hydrogen atom is replaced with an amino group.

The term “alkynyl” refers to a hydrocarbon group of 2 to 24 carbon atoms and a structural formula containing at least one carbon-carbon triple bond. The term “alkoxy” refers to a straight, branched or cyclic hydrocarbon configuration and combinations thereof, including from 1 to 20 carbon atoms, preferably from 1 to 8 carbon atoms, more preferably from 1 to 4 carbon atoms, that include an oxygen atom at the point of attachment. An example of an “alkoxy group” is represented by the formula —OR, where R can be an alkyl group, optionally substituted with an alkenyl, alkynyl, aryl, aralkyl, cycloalkyl, halogenated alkyl, or heterocycloalkyl group as described above. Suitable alkoxy groups include methoxy, ethoxy, n-propoxy, i-propoxy, n-butoxy, i-butoxy, sec-butoxy, tert-butoxy cyclopropoxy, cyclohexyloxy, and the like.

“Alkoxycarbonyl” refers to an alkoxy substituted carbonyl radical, —C(O)OR, wherein R represents an optionally substituted alkyl, aryl, aralkyl, cycloalkyl, cycloalkylalkyl or similar moiety.

The term “amine” or “amino” refers to a group of the formula —NRR′, where R and R′ can be, independently, hydrogen or an alkyl, alkenyl, alkynyl, aryl, aralkyl, cycloalkyl, halogenated alkyl, or heterocycloalkyl group described above.

“Aminocarbonyl” alone or in combination, means an amino substituted carbonyl (carbamoyl) radical, wherein the amino radical may optionally be mono- or di-substituted, such as with alkyl, aryl, aralkyl, cycloalkyl, cycloalkylalkyl, alkanoyl, alkoxycarbonyl, aralkoxycarbonyl and the like. An aminocarbonyl group may be —N(R)—C(O)—R (wherein R is a substituted group or H) or —C(O)—N(R). An “aminocarbonyl” is inclusive of an amido group. A suitable aminocarbonyl group is acetamido.

The term “amide” or “amido” is represented by the formula —C(O)NRR′, where R and R′ independently can be a hydrogen, alkyl, alkenyl, alkynyl, aryl, aralkyl, cycloalkyl, halogenated alkyl, or heterocycloalkyl group described above. A suitable amido group is acetamido.

The term “aralkyl” refers to an aryl group having an alkyl group, as defined above, attached to the aryl group, as defined above. An example of an aralkyl group is a benzyl group.

The term “aryl” refers to any carbon-based aromatic group including, but not limited to, benzene, naphthalene, etc. The term “aryl” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted.

“Carbonyl” refers to a radical of the formula —C(O)—. Carbonyl-containing groups include any substituent containing a carbon-oxygen double bond (C═O), including acyl groups, amides, carboxy groups, esters, ureas, carbamates, carbonates and ketones and aldehydes, such as substituents based on —COR or —RCHO where R is an aliphatic, heteroaliphatic, alkyl, heteroalkyl, hydroxyl, or a secondary, tertiary, or quaternary amine.

“Carboxyl” refers to a —COOH radical. Substituted carboxyl refers to —COOR where R is aliphatic, heteroaliphatic, alkyl, heteroalkyl, or a carboxylic acid or ester.

“Carcinoma” refers to any cancer that arises from epithelial cells. Carcinoma is inclusive of both malignant cancer and carcinoma in situ. Carcinoma is not inclusive of blood-borne cancers such as leukemia or myeloma.

The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, and the like. The term “heterocycloalkyl group” is a cycloalkyl group as defined above where at least one of the carbon atoms of the ring is substituted with a heteroatom such as, but not limited to, nitrogen, oxygen, sulfur, or phosphorous.

“Derivative” refers to a compound or portion of a compound that is derived from or is theoretically derivable from a parent compound.

“Drug-resistant” or “multidrug-resistant” refers to a cancer that is resistant to treatment by at least one therapeutic agent historically administered to treat that cancer. These recurrent cancers often occur after surgery, primary chemotherapy treatment, radiotherapy, or immunotherapy. In certain embodiments, the cancer is a chemotherapeutic-resistant carcinoma. For example, breast cancer may become resistant to treatment with doxorubicin, pamidronate disodium, anastrozole, exemestane, cyclophosphamide, epirubicin, raloxifene, toremifene, letrozole, trastuzumab, megastrol, tamoxifen, paclitaxel, docetaxel, capecitabine, goserelin acetate, zoledronic acid or a combination thereof.

The terms “halogenated alkyl” or “haloalkyl group” refer to an alkyl group as defined above with one or more hydrogen atoms present on these groups substituted with a halogen (F, Cl, Br, I).

The term “hydroxyl” is represented by the formula —OH.

The term “hydroxyalkyl” refers to an alkyl group that has at least one hydrogen atom substituted with a hydroxyl group. The term “alkoxyalkyl group” is defined as an alkyl group that has at least one hydrogen atom substituted with an alkoxy group described above.

“Inhibiting” (which is inclusive of “treating”) refers to inhibiting the full development of a disease or condition, for example, in a subject. “Treatment” refers to a therapeutic intervention that ameliorates a sign or symptom of a disease or pathological condition after it has begun to develop. As used herein, the term “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. “Inhibiting” also refers to any quantitative or qualitative reduction, relative to a control. A “prophylactic” treatment is a treatment administered to a subject who does not exhibit signs of a disease or exhibits only early signs for the purpose of decreasing the risk of developing pathology. By the term “coadminister” is meant that each of at least two compounds be administered during a time frame wherein the respective periods of biological activity overlap. Thus, the term includes sequential as well as coextensive administration of two or more drug compounds.

Optionally substituted groups, such as “optionally substituted alkyl,” refers to groups, such as an alkyl group, that when substituted, have from 1-5 substituents, typically 1, 2 or 3 substituents, selected from alkoxy, optionally substituted alkoxy, acyl, acylamino, acyloxy, amino, aminoacyl, aminoacyloxy, aryl, carboxyalkyl, optionally substituted cycloalkyl, optionally substituted cycloalkenyl, halogen, optionally substituted heteroaryl, optionally substituted heterocyclyl, hydroxy, sulfonyl, thiol and thioalkoxy. In particular, optionally substituted alkyl groups include, by way of example, haloalkyl groups, such as fluoroalkyl groups, including, without limitation, trifluoromethyl groups.

“Optional” or “optionally” means that the subsequently described event or circumstance can but need not occur, and that the description includes instances where said event or circumstance occurs and instances where it does not.

The terms “pharmaceutically acceptable salt or ester” refers to salts or esters prepared by conventional means that include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a hydroxy or a carboxyl group. An in vivo hydrolysable ester is an ester, which is hydrolysed in the human or animal body to produce the parent acid or alcohol. Suitable pharmaceutically acceptable esters for carboxy include C₁₋₆ alkoxymethyl esters for example methoxy-methyl, C₁₋₆ alkanoyloxymethyl esters for example pivaloyloxymethyl, phthalidyl esters, C₃₋₈ cycloalkoxycarbonyloxyC₁₋₆ alkyl esters for example 1-cyclohexylcarbonyl-oxyethyl; 1,3-dioxolen-2-onylmethyl esters for example 5-methyl-1,3-dioxolen-2-onylmethyl; and C₁₋₆ alkoxycarbonyloxyethyl esters for example 1-methoxycarbonyl-oxyethyl which may be formed at any carboxy group in the compounds.

An in vivo hydrolysable ester containing a hydroxy group includes inorganic esters such as phosphate esters and a-acyloxyalkyl ethers and related compounds which as a result of the in vivo hydrolysis of the ester breakdown to give the parent hydroxy group. Examples of a-acyloxyalkyl ethers include acetoxy-methoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

It will be appreciated that the compounds described herein may have metal binding, chelating, complex forming properties and therefore may exist as metal complexes or metal chelates.

Some of the compounds described herein may also exist in their tautomeric form.

“Saturated or unsaturated” includes substituents saturated with hydrogens, substituents completely unsaturated with hydrogens and substituents partially saturated with hydrogens.

“Selectively targeting,” as used herein, means that the agents described herein induce at least one response in targeted cancer stem cells without substantially affecting non-targeted cells, or the response in the targeted cancer stem cells is induced to a greater degree relative to the induced response in non-targeted cells. For example, the selective targeting may include selectively inhibiting proliferation of the stem cells, selectively increasing differentiation of the stem cells, selectively inducing apoptosis of the stem cells, or a combination thereof. The term “subject” includes both human and veterinary subjects.

A “therapeutically effective amount” or “diagnostically effective amount” refers to a quantity of a specified agent sufficient to achieve a desired effect in a subject being treated with that agent. Ideally, a therapeutically effective amount or diagnostically effective amount of an agent is an amount sufficient to inhibit or treat the disease without causing a substantial cytotoxic effect in the subject. The therapeutically effective amount or diagnostically effective amount of an agent will be dependent on the subject being treated, the severity of the affliction, and the manner of administration of the therapeutic composition.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. F or a general discussion of prodrugs involving esters see Svensson and Tunek, Drug Metabolism Reviews 165 (1988) and

Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester. Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions which will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS—Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

Particular examples of the presently disclosed compounds include one or more asymmetric centers; thus these compounds can exist in different stereoisomeric forms. Accordingly, compounds and compositions may be provided as individual pure enantiomers or as stereoisomeric mixtures, including racemic mixtures. In certain embodiments the compounds disclosed herein are synthesized in or are purified to be in substantially enantiopure form, such as in a 90% enantiomeric excess, a 95% enantiomeric excess, a 97% enantiomeric excess or even in greater than a 99% enantiomeric excess, such as in enantiopure form.

### Anti-Cancer Agents

The agents describe herein may exhibit activity for selectively targeting cancer stem cells. Illustrative agents include purine compounds, phenolic compounds, and rottlerin compounds. In certain embodiments, the agents may be low molecular weight compounds (“LMWCs”, having a molecular weight of less than about, for example and not by way of limitation, 600 daltons).

An example of a purine compound is a substituted purine compound wherein at least one ring position of the purine heterocyclic structure is substituted with at least one moiety (other than H). For example, the substituted purine compound may have a structure represented by:

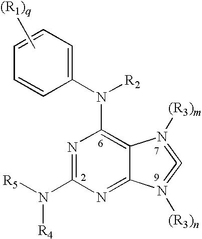

in which q is 1-5,

R₁ is halogen, lower alkyl, hydroxyl or lower alkanoyloxy; lower alkoxy which is unsubstituted or substituted by hydroxyl, lower alkoxy or carboxyl; a radical of the formula —O(—CH₂—CH₂—O)ₜ—R₆, in which t is 2-5 and R₆ is hydrogen or lower alkyl; carboxyl, lower alkoxycarbonyl, piperazin-1-yl-carbonyl or carbamoyl; N-lower alkyl-carbamoyl, which is unsubstituted or substituted by hydroxyl or amino in the lower alkyl moiety; N,N-di-lower alkyl-carbamoyl, cyano, nitro, amino, lower alkanoyl amino, lower alkylamino, N,N-di-lower alkylamino, aminosulfonyl or trifluoromethyl, where, if more than one radical R is present in the molecule, these can be identical to or different from one another,

R₂ is hydrogen, carbamoyl or N-lower alkyl-carbamoyl, m and n are each 0 or 1, where m is 0 if n is 1 and m is 1 if n is 0,

R₃ is lower alkyl or phenyl which are unsubstituted or in each case substituted by hydroxyl, lower alkoxy, amino, lower alkylamino or N,N-di-lower alkyl amino, and

a) R₄ is hydrogen, amino, phenylamino, lower alkylamino, hydroxyl, phenoxy, lower alkoxy, acyl having 1-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms and R₅ is amino, phenylamino, lower alkylamino, hydroxyl, phenoxy, lower alkoxy, acyl having 2-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms, or
b) R₄ and R₅ together are a substituted or unsubstituted alkylene or alkenylene radical having in each case not more than 15 C atoms, in which 1-3 C atoms can be replaced by oxygen, sulfur or nitrogen, and their salts.

The formula above is inclusive of the formulae below derived from the corresponding tautomeric purine derivatives, in which the symbols are as defined above.

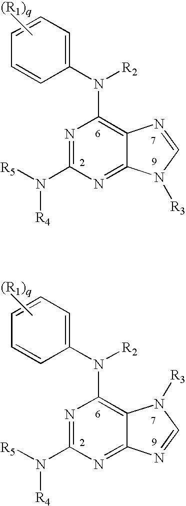

In certain embodiments, q is 1,
R₁ is chlorine which is in the 3 position,
R₂ is hydrogen, m is 0 and n is 1,
R₃ is ethyl and a) R₄ is hydrogen and
R₅ is amino, phenylamino, lower alkylamino, hydroxyl, phenoxy, lower alkoxy; an acyl radical of the part formula Z—C(═W)—, in which W is oxygen, sulfur or imino and Z is hydrogen, hydrocarbyl R^{o}, hydrocarbyloxy R^{o}—O— or an amino group of the formula R₇(R₈)N—, in which R^{o} in each case is C₁-C₄ alkyl, hydroxylC₂-C₁₄alkyl, cyano-C₁-C₄alkyl, carboxy-C₁-C₄alkyl, C₁-C₄alkoxycarbonyl-C₁-C₄alkyl, C₃-C₇alkenyl or phenyl and R₇ and R₈ independently of one another are each hydrogen, lower alkyl, ω-amino-lower alkyl, lower alkylsulfonyl or phenyl;
2-carbamoyl-1-carboxy-eth-1-yl, 3-amino-2-hydroxy-prop-1-yl, 3-amino-prop-1-yl, 3-amino-2,2-dimethyl-prop-1-yl, 3-amino-2-oxo-prop-1-yl, 3-amino-1-carboxy-prop-1-yl, 3-amino-3-carboxy-prop-1-yl, 1,1-dicarbamoyl-methyl, 2-carbamoyl-eth-1-yl, 3-amino-1,3-di-hydroxyl-imino-prop-1-yl, 2-carbamoyl-1-hydroxylimino-eth-1-yl, 1-hydroxylimino-2-thiocarbamoyl-eth-1-yl, 3-amino-3-hydroxylimino-1-thio-prop-1-yl, 3-amino-pent-1-yl, 1-amino-pent-3-yl, 1-amidino-1-carbamoyl-methyl, 4-amino-1,1,1,3,5,5,5-heptafluoro-pent-2-yl, 3-amino-1,3-dicarboxy-prop-1-yl, 2-carbamoyl-1-ethoxycarbonyl-eth-1-yl, 2-amino-1,2-dithio-eth-1-yl, 2-amino-1,2-dioxo-eth-1-yl, 2-amino-2-methyl-prop-1-yl, 1-amino-2-methyl-prop-2-yl, 2-amino-prop-1-yl, 1-amino-prop-2-yl, 2-amino-eth-1-yl, 2-amino-2-carboxy-eth-1-yl, 2-amino-1-carboxy-eth-1-yl, carbamoyl-methyl, 1-carbamoyl-3-methyl-but-1-yl, 2-amino-1,2-dicarboxy-eth-1-yl, 1-carbamoyl-3-methylthio-prop-1-yl, 1-carbamoyl-2-methyl-prop-1-yl, 1-carbamoyl-eth-1-yl, 1-carbamoyl-1-cyano-methyl, 1-carbamoyl-3-carboxy-3-fluoro-prop-1-yl, 1-carbamoyl-2-carboxy-eth-1-yl, 2-amino-4-carboxy-but-1-yl, 1-amino-4-carboxy-but-2-yl, 1-carbamoyl-4-guanidino-but-1-yl, 1-carbamoyl-5-amino-pent-1-yl, 1-carbamoyl-2-hydroxy-prop-1-yl, 1-carbamoyl-2-methyl-but-1-yl, 1-carbamoyl-2-hydroxy-eth-1-yl, 1,3-dicarbamoyl-prop-1-yl, 2-amino-but-1-yl, 1-amino-but-2-yl, 1-carbamoyl-pent-1-yl, 1-carbamoyl-but-1-yl; benzyl, 2-phenyl-ethyl, 3-aminomethyl-benzyl, (1-hydroxy-cyclohex-1-yl)-methyl, (2-amino-3,5,5-trimethyl-cyclopentyl)-methyl, 1-[N-(1-carboxy-2-phenyl-ethyl)-carbamoyl]-2-carbamoyl-eth-1-yl, 1-carbamoyl-1-phenyl-methyl, 1-carbamoyl-2-(4-hydroxy-phenyl)-eth-1-yl, 1-carbamoyl-2-phenyl-eth-1-yl, 2-amino-1,2-diphenyl-eth-1-yl, 2-benzyloxycarbonyl-1-carbamoyl-etn-1-yl, 3-benzyloxycarbonyl-1-carbamoyl-prop-1-yl, 1-adamantyl-2-amino-prop-1-yl, 1-adamantyl-1-amino-prop-2-yl, (2-furyl)-methyl, (2-tetrahydrofuryl)-methyl, 2-pyrid-2-yl-ethyl, 2-piperidino-ethyl, 2-(morpho-lin-4-yl)-ethyl, 2-(3-indolyl)-ethyl, 2-(4-imidazolyl)-ethyl, 1-carbamoyl-2-β-indolyl)-eth-1-yl, 1-carbamoyl-2-imidazol-4-yl-eth-1-yl, 1-carbamoyl-2-indol-3-yl-eth-1-yl, 3-aminomethyl-oxetan-3-yl-methyl, 1-(acetoxy-imino)-1-(4-amino-2-oxa-1,3-diazol-5-yl)-methyl, 2-amino-cyclohex-1-yl, 3-amino-cyclohex-1-yl, 2-aminomethyl-3,3,5-trimethyl-cyclopent-1-yl, 3-amino-adamantan-1-yl, 2-carbamoyl-bicyclo[2.2.1]hept-5-en-3-yl, 2-carbamoyl-cyclohex-1-yl, 9-amino-spiro-[4.4]non-1-yl,
5-amino-2-oxa-1,3-diazol-4-yl, 4-amino-thien-3-yl, 3-carbamoyl-5-(3-[2,4-dichloro-phenyl]-1-oxo-prop-2-en-1-yl)-1,2-thiazol-4-yl, 3-carbamoyl-5-(3-[4-trifluoro-phenyl]-1-oxo-prop-2-en-1-yl)-1,2-thiazol-4-yl, 4-amino-2-(4-carboxy-butyl)-tetrahydrothiophen-3-yl, 3-amino-2-(4-carboxy-butyl)-tetrahydrothiophen-4-yl, [1,2,5]oxadiazolo[3,4-b](6-amino-pyrazin-5-yl), 2,5′-diacetyl-3-amino-thieno[2,3-b]thiophen-4′-yl or 3-amino-2,5*-dipivaloyl-thieno[2,3-b]thiophen-4′-yl, or
   b) R₄ and R₅ together are 1 ,2-ethylene, propane-1,3-diyl, butane-1,4-diyl, pentane-1,5-diyl, 3-(3-amino-propionyl)-3-aza-pentane-1,5-diyl, 1-aminomethyl-butane-1,4-diyl, 1-hydroxy-methyl-butane-1,4-diyl, 3-(2-amino-ethyl)-pentane-1,5-diyl, 3-aza-pentane-1,5-diyl or 3-(2-amino-ethyl)-3-aza-pentane-1,5-diyl, or a salt thereof.

Examples of such substituted purine compounds are disclosed in WO 1997/16452, which is incorporated herein by reference. A specific purine compound useful as a cancer stem cell-selective agent is N-(cis-2-aminocyclohexyl)-N-(3-chlorophenyl)-9-ethyl-9H-purine-2,6-diamine (also known as CGP74514A available from Calbiochem) which has the structure:

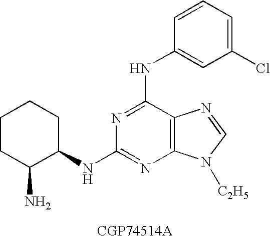

The phenolic compound may have a structure represented by:

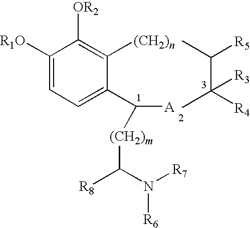

wherein A is O, C, CH, or CH₂;
R₁ and R₂ are independently hydrogen or a leaving group or a protecting group;
m and n are independently selected from zero or 1;
R₃ is H, alkyl, alkenyl, aryl, cycloalkyl, or taken together with R₄ can form a spirocycloalkyl, with the proviso that when n is zero R₃ is not H;
R₄ is H or alkyl, or taken together with R₃ can form a spirocycloalkyl;
R₅ is H or alkyl, or when n is zero, R₅ can be taken together with R₃ to form a fused cycloalkyl;
R₆ is H, alkyl, or taken together with R₈ can form an N containing heterocycle;
R₇ is H, alkyl, alkenyl, cycloalkyl, arylalkyl, or taken together with A when A is C and when m=0 and n=0, can form a fused N containing heterocycle, or taken together with R₈ can form an N containing heterocycle; or R₆ and R₇ together can form an N containing heterocycle with the proviso that when R₆ is alkyl R₇ cannot be arylalkyl; R₈ is H, alkyl, taken together with R₆ or R₇ to form an N containing heterocycle, or taken together with the catechol ring can form a fused ring;
or pharmaceutically acceptable salts, esters or amides thereof.

Examples of phenolic compounds are disclosed in U.S. Pat. No. 4,963,568, which is incorporated herein by reference. In one embodiment, the phenolic compound is [1R,3S]3-(1′-adamantyl)-1-aminomethyl-3,4-dihydro-5,6-dihydroxy-1H-2-benzopyran hydrochloride hydrate) (commercially available as A-77636), as shown below.

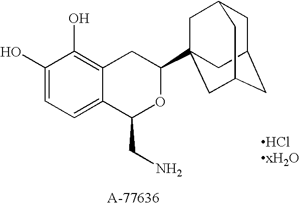

The rottlerin compound may have a structure represented by:

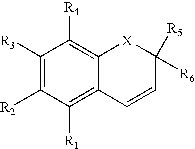

wherein X is selected from the group consisting of CH₂, O, N, and S; R₁ and R₃ are selected from the group consisting of H, OH, NH and SH; R₂ is selected from the group consisting of ethanone, acetyl, alkenyl, aryl and alkyl; R₄ is CO—[CHCH]ₙ-Ph, CN—[CHCH]ₙ-Ph, or COOZ, wherein Z is selected from the group consisting of alkenyl, aryl, and alkyl; R₅ and R₆ are selected from the group consisting of H, OH, NH, SH, alkenyl, aryl, and alkyl; and n is 0 to 5.

Examples of rottlerin compounds are disclosed in WO 2006/060196, which is incorporated herein by reference. In one embodiment, the agent is rottlerin (mallotoxin; 5,7-dihydroxy-2,2-dimethyl-6-(2,4,6-trihydroxy-3-methyl-5-acetylbenzyl)-8-cinnamoyl-1,2-chromene), a natural product from Mallotus phillippinensis, as shown below.

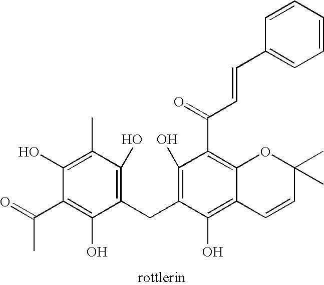

### Composition and Methods

The compounds and pharmaceutical compositions disclosed herein can be used for inhibiting or treating cancer, particularly drug-resistant cancer. In particular, the agents disclosed herein may selectively target cancer, particularly carcinoma, stem cells. The cancer stem cells may be present in, or obtained from, a solid tumor.

Illustrative carcinoma stem cells that may be targeted include, for example, but not by way of limitation, breast cancer, prostate cancer, glioblastoma, colon carcinoma, lung carcinoma, pancreatic cancer, melanoma, gastric cancer, hepatic carcinoma, ovarian carcinoma, and testicular cancer. Other cancer stem cells for targeting include lymphoma, and leukemia.

The preparation and use of immortalized cancer stem cells is described in commonly-assigned U.S. Provisional Application 61/052,846, “Cancer Stem Cell Immortalization”, filed May 13, 2008, which is incorporated herein by reference in its entirety. Introduction of an Oct3/4 promoter sequence was observed to stabilize the undifferentiated phenotype of cancer stem cells. This effect is alternatively referred to herein as “immortalization”, which as defined herein, does not require that a culture of such cells would persist indefinitely. Similarly, stability of the undifferentiated phenotype of cancer stem cells, as that phrase is used herein, does not require that all aspects of the cancer stem cell phenotype be retained, but rather that at least one or more characteristics of that phenotype be retained (although not necessarily at the same level as native cells). For example, one or more of the following characteristics are retained: the expression of one or more surface antigen; the level of expression of one or more surface antigen; permeability to a histologic dye; number of cells required to produce a tumor when implanted into a host animal; characteristics of tumors produced from the cells; morphology in culture; association with other cells in culture; and sensitivity to pharmacologic agents.

“Oct3/4 promoter sequences” are a family of sequences which are related to the native promoter operably linked to an Oct3/4 gene. In one embodiment, the Oct3/4 gene is the human Oct3/4 gene. A variety of lengths of sequences are encompassed within the “Oct3/4 promoter sequences” family. Likewise, some of the sequences exhibit demonstrable promoter activity (for example, of the Oct3/4 gene and/or a reporter gene), whereas others do not. Members of the “Oct3/4 promoter family”, share that property that, when introduced into a cancer stem cell, they promote the persistence of the cancer stem cell phenotype.

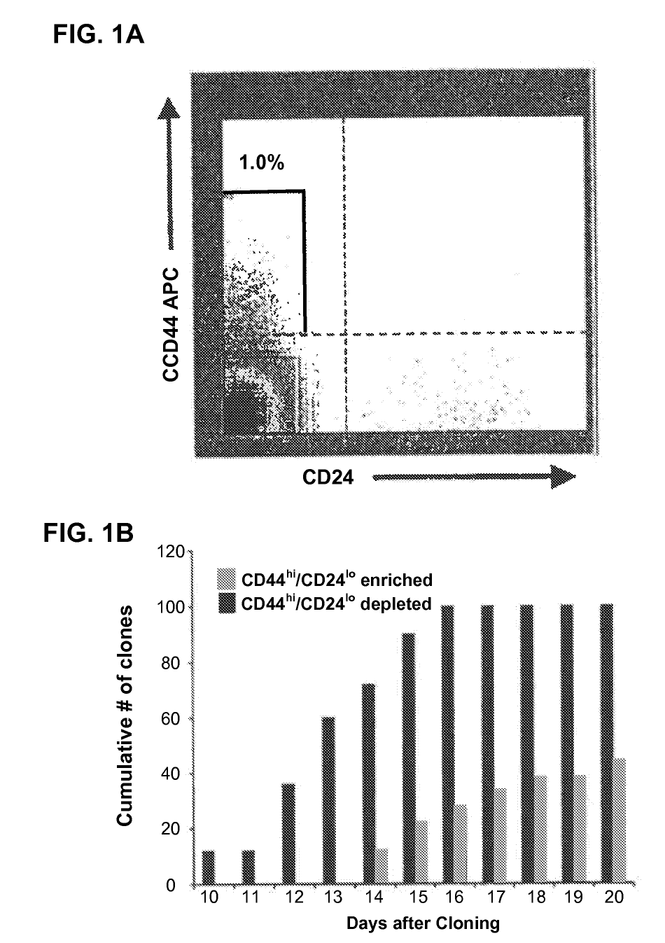

In one specific, non-limiting embodiment, the Oct3/4 promoter sequence is the human sequence set forth in FIG. 1 (GenBank Acc. No. DQ249177) (SEQ ID NO:1), or a sequence which is at least 90 percent, homologous thereto as determined by standard programs, such as BLAST or FASTA.

In other non-limiting embodiments, the Oct3/4 promoter sequence does not contain the entire sequence depicted in FIG. 1, but rather a subsequence thereof that, when introduced into a cancer stem cell, promotes the persistence of the cancer stem cell phenotype. In various non-limiting embodiments, the Oct3/4 promoter sequence is such a subsequence (which is not the entire sequence) which comprises virtual ORF1 (ca. 1.1-1.6 kb from the 5′ end or a sequence at least 90 percent or at least 95 percent homologous thereto. which could potentially encode a 162 amino acid protein), or virtual ORF2 (ca. 2.4-2.8 kb from the 5′ end or a sequence at least 90 percent or at least 95 percent homologous thereto, which could encode a 138 amino acid protein) or virtual ORF3 (ca. 2.7-3.3 kb from the 5′ end or a sequence at least 90 percent or at least 95 percent homologous thereto, which could encode a 201 amino acid protein), or some combination thereof, such as two of the three virtual ORF. Boundaries of the 3 ORFs in the Oct3/4 promoter, with translational start and termination sites underlined, are set forth below. These sequences may be comprised in primers for polymerase chain reaction (“PCR”) to amplify the ORF in expression form.

| ORF138 (Starts with CTG) | |
|---|---|
| FWD: | |
| TGG AAC CTG CAC ATC AGG TTC C | (SEQ ID NO: 2) |
| REV: | |
| TCA GTC TTT GAG GGG ATT GCA | (SEQ ID NO: 3) |
| ORF201 (Starts with CTG) | |
| FWD: | |
| TCC CAG CTG TCT GGA ATC ACT CC | (SEQ ID NO: 4) |
| REV: | |
| CTA CCG TGG TAT TAG ATG TCT G | (SEQ ID NO: 5) |
| ORF162 (Starts with ATG) | |
| FWD: | |
| TAG ATT ATG GGG CCT GGT GG | (SEQ ID NO: 6) |
| REV: | |
| CTA GGA GTC TAG GCA TGC AGG. | (SEQ ID NO: 7) |

In other non-limiting embodiments, the Oct/34 promoter sequence is a subsequence resulting from cleavage of SEQ NO:1 at the NdeI site shown in FIG. 9 (either the resulting 5′ fragment or the 3′ fragment, or a sequence at least 90 percent or at least 95 percent homologous thereto); or resulting from cleavage of SEQ NO:1 at the Sail site shown in FIG. 9 (either the resulting 5′ fragment or the resulting 3′ fragment, or a sequence at least 90 percent or at least 95 percent homologous thereto); or resulting from cleavage of SEQ NO:1 at the Sad site shown in FIG. 9 (either the resulting 5′ fragment or the resulting 3′ fragment, or a sequence at least 90 percent or at least 95 percent homologous thereto); or resulting from cleavage of SEQU NO:1 at the AccI site shown in FIG. 9 (either the resulting 5′ fragment or the resulting 3′ fragment, or a sequence at least 90 percent or at least 95 percent homologous thereto); or resulting from cleavage of SEQ NO:1 at the EcoRI site shown in FIG. 9 (either the resulting 5′ fragment or the resulting 3′ fragment, or a sequence at least 90 percent or at least 95 percent homologous thereto).

In other non-limiting embodiments, an Oct3/4 promoter sequence is a subsequence of SEQ ID NO:1 consisting essentially of at least 250, or at least 500, or at least 750, or at least 1000, or at least 1250, or at least 1500, or at least 1750, or at least 2000, or at least 2250, or at least 2500, or at least 2750, or at least 3000, or at least 3250, or at least 3500 contiguous residues of SEQ ID NO:1 or a sequence at least 90 percent or at least 95 percent or at least 98 percent homologous thereto.

In further non-limiting embodiments, an Oct3/4 promoter sequence is a subsequence of SEQ ID NO:1, or a sequence at least 90 percent or at least 95 percent or at least 98 percent homologous thereto, which lacks at least 250, or at least 500, or at least 750, or at least 1000, or at least 1250, or at least 1500, or at least 1750, or at least 2000, or at least 2250, or at least 2500, or at least 2750, or at least 3000, or at least 3250, or at least 3500 contiguous residues of SEQ ID NO:1.

In one specific, non-limiting embodiment, the Oct3/4 promoter sequence is the murine sequence set forth in FIG. 10 (SEQ ID NO:8), or a sequence which is at least 90 percent, or at least 95 percent, homologous thereto as determined by standard programs, such as BLAST or FASTA.

In other non-limiting embodiments, the Oct3/4 promoter sequence does not contain the entire sequence depicted in FIG. 10, but rather a subsequence thereof that, when introduced into a cancer stem cell, promotes the persistence of the cancer stem cell phenotype.

In other non-limiting embodiments, an Oct3/4 promoter sequence is a subsequence of SEQ ID NO:8 consisting essentially of at least 250, or at least 500, or at least 750, or at least 1000, or at least 1250, or at least 1500, or at least 1750, contiguous residues of SEQ ID NO:8 or a sequence at least 90 percent or at least 95 percent or at least 98 percent homologous thereto.

In further non-limiting embodiments, an Oct3/4 promoter sequence is a subsequence of SEQ ID NO:8, or a sequence at least 90 percent or at least 95 percent or at least 98 percent homologous thereto, which lacks at least 250, or at least 500, or at least 750, or at least 1000, or at least 1250, or at least 1500, or at least 1750 contiguous residues of SEQ ID NO:8.

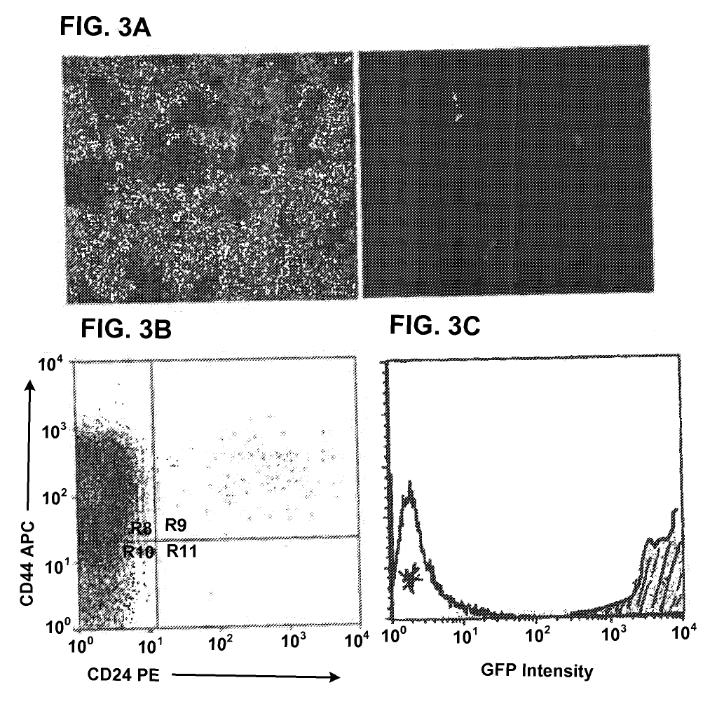

An Oct3/4 promoter sequence for use according to the invention may be identified by using standard techniques to introduce said promoter sequence, optionally operably linked to a reporter gene, into a cancer stem cell, such as a cancer stem cell from a MCF7 cell population, and then determining whether introduction of said sequence results in stabilization of the cancer stem cell phenotype. In a specific, non-limiting example, a putative Oct3/4 promoter sequence may be linearized in a plasmid backbone comprising Neo as a selection marker and electroporated into ca. 5×10⁶ MCF7 cells. The cells may then be selected in G-418 as described in the legend to FIG. 3. Pooled G418-resistant colonies may then be incubated with mAbs directed against CD44 and CD24 and the CD44hi/CD24lo CSC population may be isolated by cell sorting. The collected cells may then be cultured and the level of stability of the stem cell phenotype may be assessed.

An Oct3/4 promoter sequence as described in the foregoing section may be comprised in a construct for introduction into a cancer stem cell. In certain, but not all, embodiments, said construct further comprises a reporter gene operably linked to the Oct3/4 promoter sequence.

Said construct may be comprised in a vector, such as, but not limited to, a plasmid, a phage, a cosmid, or a virus. Non-limiting examples of viruses which may serve as vectors include lentivirus vectors such as retroviruses, adenovirus, vaccinia virus, adenoassociated virus, etc. Said vector may optionally comprise a gene encoding a selectable product (for example (but not by way of limitation) that confers resistance to an antibiotic) distinct from the reporter gene.

Any reporter gene, namely a gene that encodes a detectable product, may be used. Preferably the product is detectable in vivo, so that a cancer stem cell containing the construct may be collected based on reporter gene expression and remain viable. Non-limiting examples of suitable reporter genes include a fluorescent protein, such as a Green Fluorescent Protein

(“GFP”, enhanced Green Fluorescent Protein (“eGFP”), a Yellow Fluorescent Protein, a Red Fluorescent Protein, etc., a gene that confers antibiotic resistance, or a gene that encodes a cell surface protein no typically found on the cancer stem cell surface or any gene that encodes a product which confers a detectable phenotype.

In particular non-limiting embodiments, the construct is an isolated nucleic acid comprising an Oct3/4 promoter sequence operably linked to a reporter gene, wherein the Oct3/4 promoter sequence is essentially the entire sequence set forth in FIG. 9 (GenBank Acc. No. DQ249177) (SEQ ID NO:1), or a sequence at least 90 percent, or at least 95 percent, homologous thereto. Said nucleic acid may be contained in a vector.

In other particular non-limiting embodiments, the construct is an isolated nucleic acid comprising an Oct3/4 promoter sequence operably linked to a reporter gene, wherein the Oct3/4 promoter sequence does not contain the entire sequence set forth in FIG. 9 (GenBank Acc. No. DQ249177) (SEQ ID NO:1) (see the preceding section). Said nucleic acid may be comprised in a vector.

In still further non-limiting embodiments, the construct is an isolated nucleic acid which may be used to reversibly immortalize a cancer stem cell, comprising an Oct3/4 promoter sequence operably linked to a reporter gene, wherein said promoter linked to the gene is flanked by lox sites. Said construct may be comprised in a vector. The use of such a construct is discussed in detail below.

In still further non-limiting embodiments, the construct is an isolated nucleic acid comprising an Oct3/4 promoter sequence, and further comprising a gene encoding a selectable marker operably linked to a promoter sequence selectively active in a cancer stem cell (but much less active in a transient amplifying cell), for example a promoter associated with genes such as Cripto, gastrin-releasing peptide receptor, procalyxin-like protein, and hTERT.

A cancer stem cell obtained from any type of cancer may be immortalized—its phenotype stabilized. The cancer stem cell may be from a human or a non-human subject. The cancer stem cell may be obtained from a tumor cell line or for a primary tumor.

A cancer stem cell may be collected by any means known in the art. For example, a cancer stem cell may be collected from (isolated from or enriched from) a larger population of cells using cell surface markers or other properties typical to that cancer stem cell. Alternatively, an Oct3/4 promoter sequence-containing construct which contains a selectable marker which is selectively expressed in a cancer stem cell may be collected from the population via the selectable marker, for example by fluorescence activated cell sorting (“FACS”).

Expression of the ALDH1 isoform (9a) may be used as a cancer stem cell marker. For example, the Aldefluor Assay (Stem Cell Technologies, Inc.) may be used. In non-limiting embodiments, where the cancer stem cell is a breast cancer stem cell, a phenotype of cell marker expression CD44hi (meaning increased relative to normal control) and CD24lo (meaning decreased relative to normal control) may be used to collect cancer stem cells (for example, using antibodies directed to said proteins and FACS). Where a cell line is used as the source of cancer stem cells, suitable cell lines include, but are not limited to MCF7, T-47D, UACC-812, HCC38, HCC1428, SKBR-3, and MB-157.

In non-limiting embodiments, where the cancer stem cell is a colon cancer stem cell, a phenotype of cell marker expression EpCAMhi/CD44hi, or expression of CD133, or the ability to exclude the dye Hoechst 33342, may be used to collect cancer stem cells. Where a cell line is used as the source of cancer stem cells, suitable cell lines include, but are not limited to Colo320, HCT15, and SW480.

In non-limiting embodiments, where the cancer stem cell is a prostate cancer stem cell, a phenotype of cell marker expression CD44hiCD24lo/Sca1+ or the ability to exclude the dye Hoechst 22243, may be used to collect cancer stem cells. Where a cell line is used as the source of cancer stem cells, suitable cell lines include, but are not limited to PC3, DU145, and LNCaP. In non-limiting embodiments, where the cancer stem cell is a pancreatic cancer stem cell, a phenotype of cell marker expression CD44hi, CD24hi, ESAhi may be used to collect cancer stem cells. Where a cell line is used as the source of cancer stem cells, suitable cell lines include, but are not limited to PANC-1 and ASPC-1.

The immortalized cancer stem cells may be used to identify useful therapeutic agents, by screening various test agents. The test agents may be known bioactive compounds or may be compounds without hitherto known biological activity. Suitable test agents may also be biologic molecules, including but not limited to proteins, antibodies or antibody fragments, oligonucleotides, peptidomimetic compounds, etc.

One method of identifying an anti-cancer agent includes: providing an isolated cancer stem cell containing an Oct3/4 promoter sequence which is not operably linked to an Oct3/4 gene;

(ii) providing a means for evaluating the proliferation, differentiation level, and/or viability of the cancer stem cell;

(iii) administering a test agent to the cancer stem cell; and

(iv) evaluating the proliferation and/or differentiation and/or viability of the cancer stem cell;

wherein an inhibition of proliferation, increase in level of differentiation, or decrease in viability associated with the presence of the test agent indicates that the test agent is an anti-cancer agent. In certain non-limiting embodiments of this method, the means for evaluating the proliferation, differentiation level, and/or viability comprising measuring and/or detecting expression of a reporter gene (see above).

In certain embodiments, a method of identifying an anti-cancer agent with selective activity toward cancer stem cells includes:

(i) providing a population of cancer cells comprising cancer stem cells as well as cancer cells which are not stem cells, where the relative proportions of cancer stem cells and cancer cells which are not stem cells is known;

(ii) administering a test agent to the population of cells;

(iii) culturing the population after (ii); and

(iv) determining the relative proportions of cancer stem cells and cancer cells which are not stem cells in the population after (iii);

wherein a decrease in the relative proportion of cancer stem cells indicates that the test agent is an anti-cancer agent with selective activity against cancer stem cells. In non-limiting embodiments of this method, there is a first means for detecting a cancer stem cell and a second, different means for detecting a cancer cell that is not a stem cell, where said first means and said second means are used to determine the relative proportions of cancer stem cells and cancer cells which are not stem cells. For example, in non-limiting embodiments said first means and/or second means may be a fluorescent antibody to a cell surface antigen (if both means are fluorescent antibodies, they are desirably of different colors). Alternatively, in other non-limiting embodiments, said first means and/or second means may be an expression construct having a reporter gene selectively expressed in a cancer stem cell or a cancer cell which is not a stem cell (if both means are reporter genes, they preferably encode different products). As a specific non-limiting example, a cancer stem cell may be detected via a detectable reporter gene (e.g. a fluorescent protein of a first color) operably linked to an Oct3/4 promoter sequence with promoter activity, and a cancer cell which is not a stem cell may be detected by a fluorescent antibody (of a second color) which recognizes a surface antigen on said cancer cell but absent or in substantially lower amounts on a cancer stem cell.

In non-limiting embodiments, high throughput screening techniques may be used.

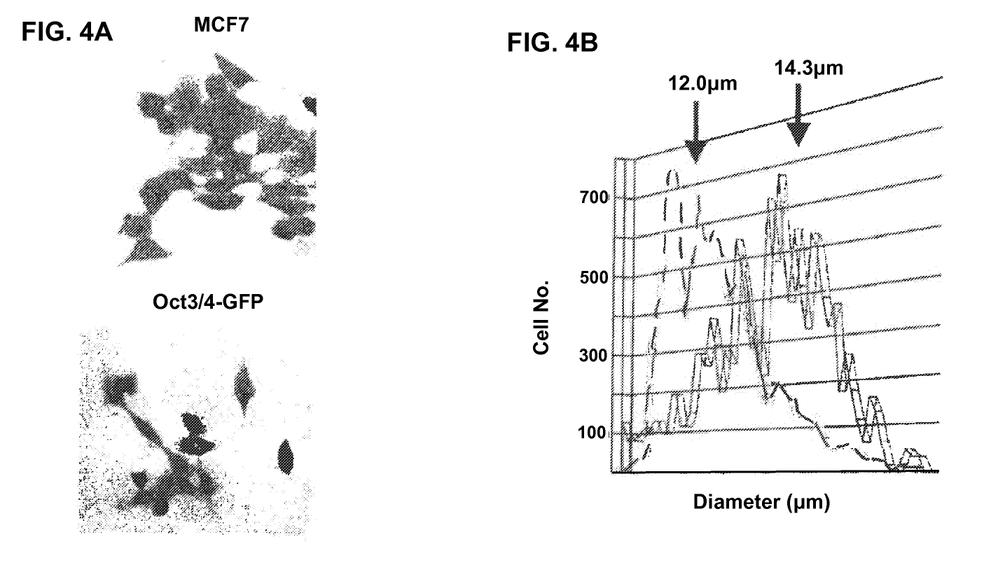

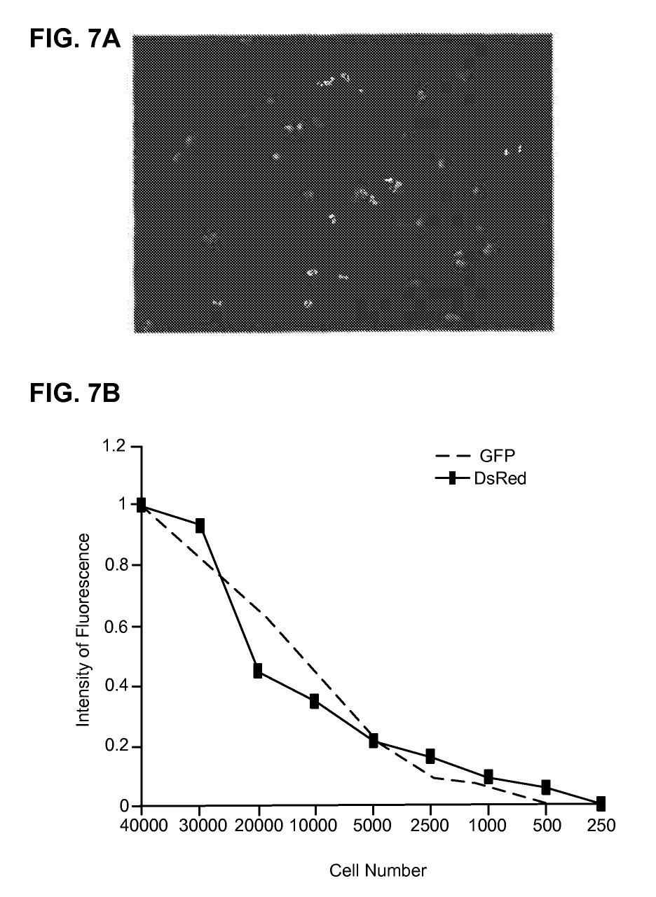

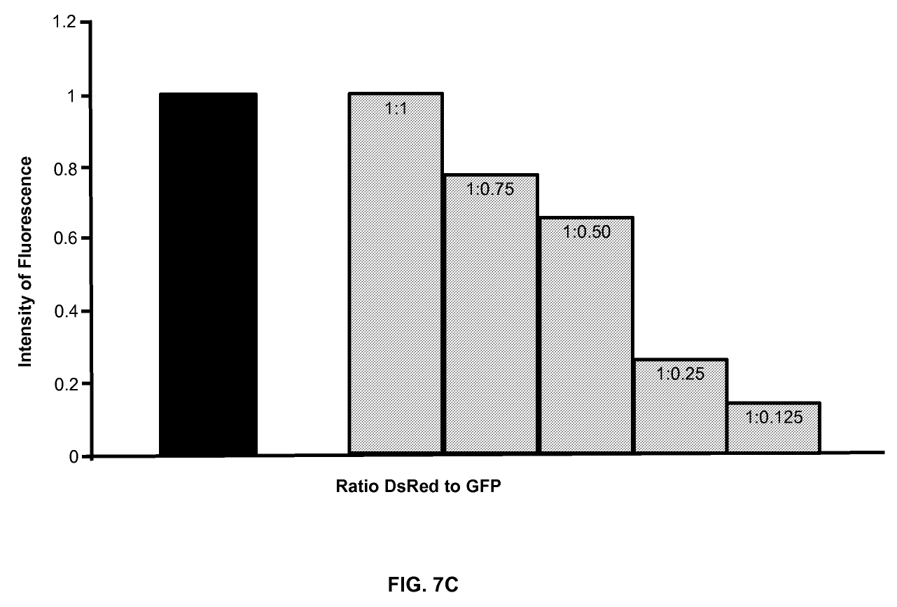

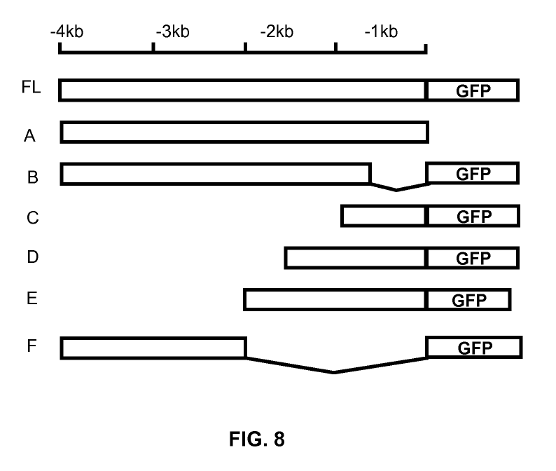

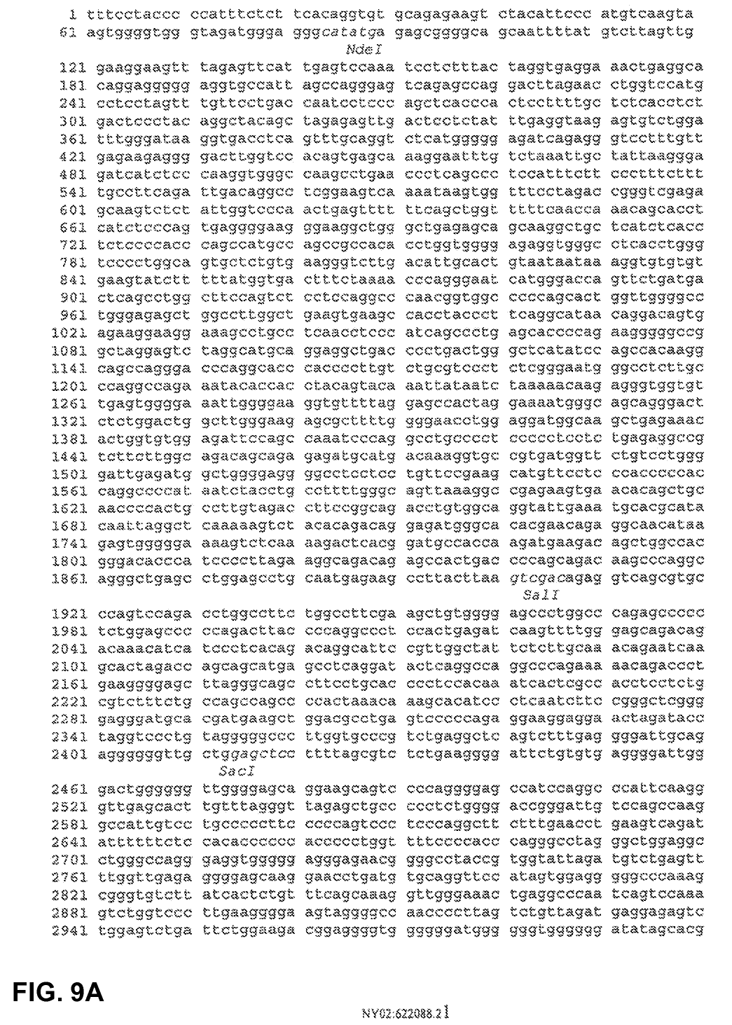

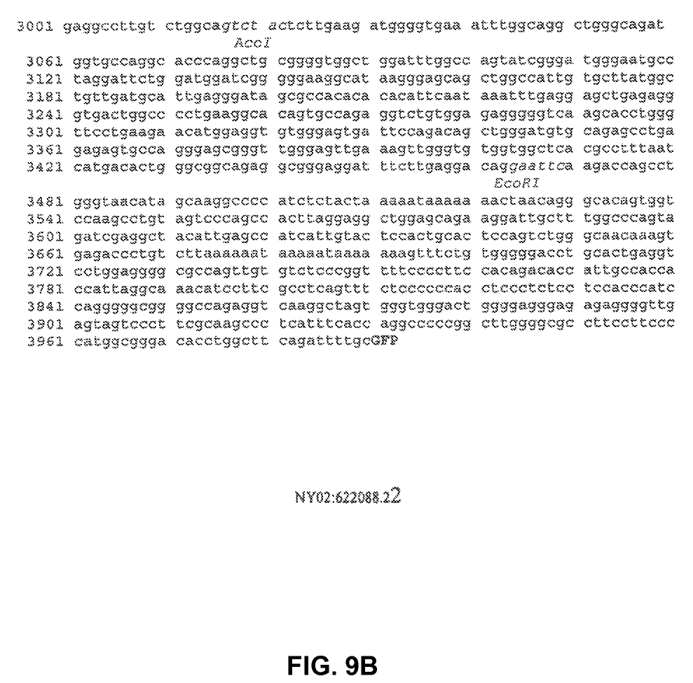

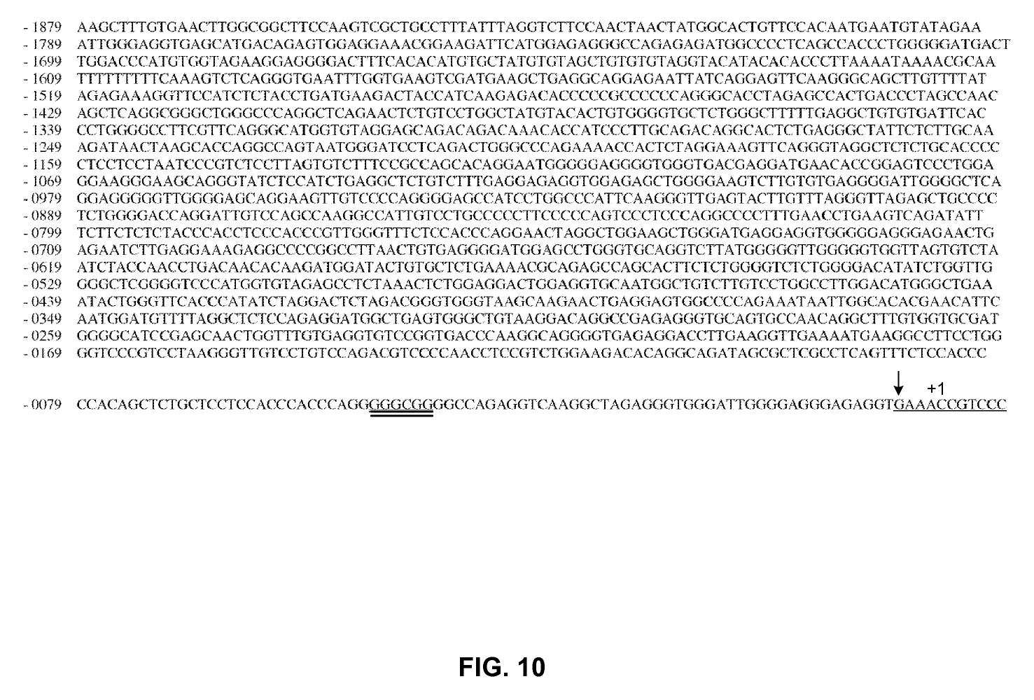

In a specific, non-limiting example, an assay for screening for anti-cancer agents is described as follows. Unfractionated, non-CSC-MCF7 cells may be infected with a lentiviral vector encoding DsRED (Clontech). CSC-MCF7 cells may separately be transfected or infected with an expression construct comprising an Oct3/4 promoter operably linked to GFP. In the assay, LMWC may be identified which inhibit proliferation/survival of GFP+CSCs relative to control DsRED-tagged MCF7 cells. The screening procedure may use a 1:1 mix of the above-described GFP and DsRED cells. After robotically dispensing a total of ˜2000 cells into 384 well plates, they may be incubated overnight to allow attachment. LMWCs (each in DMSO) may then be added to each well to a final compound concentration of 10 μM (final DMSO concentration<1% which is easily tolerated). GFP:DsRED ratios may then be determined daily over the ensuing 2-3 days (see FIG. 7). All compounds that reduce GFP:DsRED ratios>3SDs below the mean of the control (No LMWC added; DMSO vehicle only) may be flagged for subsequent follow up. Further refinement of the “hits” may utilize advanced mathematical methods such as B-scores and BZ-scores to reduce the false positive rate even lower. The foregoing assay, while sensitive, may lack specificity as there are several ways that a compound could alter the GFP:DsRED ratio other than by inhibiting the proliferation or survival of the GFP+population: for example, (i) it could inhibit or quench the fluorescence intensity of GFP (specific or non-specific but not of interest); (ii) it could promote the growth of the DsRED population (specific but likely not of interest); (iii) it could enhance the fluorescence of DsRED (specific or non-specific but not of interest); or (iv) it could promote the differentiation of the GFP+population into TACs associated with concurrent down-regulation of the Oct3/4 promoter (specific and of interest). Most of these possibilities would not be expected to be distinguishable on the initial screen. Therefore, a compounds flagged in this “first pass” assay would preferably be subjected to further testing, for example re-testing in triplicate. Repeat hits may then be examined at serial dilutions to establish ID50's and to identify compounds that are active at submicromolar concentrations. The lowest concentration of compounds that maximally inhibit the GFP signal may then be re-screened with isolated populations of Oct3/4-GFP+MCF7, DsRED-MCF7 cells and an additional line of MCF7 cells that expresses GFP under the control of a neutral (CMV) promoter. Growth curves for each population may be determined, and apoptosis assays (TUNEL assays, Annexin V staining, and/or caspase-3 cleavage [Caspase3/7, Promega]) may be performed. Visual inspection of cells, flow cytometry and cell sizing using a Vi-Cell apparatus (FIG. 4) may be used to determine whether loss of GFP expression in individual cells is occurring as would be expected if the compound were promoting CSC differentiation that might, without affecting cell proliferation or viability, cause loss of GFP due to a differentiation-mediated down-regulation of the Oct3/4 promoter. H&E and CD44/CD24 staining may be used to confirm this by documenting changes in morphology and cell surface phenotype.

Also disclosed is a means of identifying an agent likely to be of benefit to a subject, where the subject has a cancer, comprising:

(i) collecting a cancer stem cell from the subject;

(ii) introducing, into the cancer stem cell from the subject, a nucleic acid comprising as Oct3/4 promoter sequence operably linked to a reporter gene;

(iii) exposing the product of step (ii) to an agent; and

(iv) determining whether exposure to the agent inhibits proliferation, increases the level of differentiation, and/or decreases the viability of the product of step (ii),

where an inhibition of proliferation, increase in the level of differentiation, or decrease of viability indicates that the agent may be of therapeutic benefit to the subject. This method may be desirably practiced for a number of different agents, and selecting the agent which most effectively, among those tested, inhibits proliferation, increases the level of differentiation, and/or decreases viability of the cancer stem cell containing the nucleic acid comprising an Oct3/4 promoter sequence operably linked to a reporter gene.

In further embodiments, the compounds disclosed herein may be co-administered with another pharmaceutically active compound. For example, the compounds may be co-administered with another anti-cancer compound such as doxorubicin, pamidronate disodium, anastrozole, exemestane, cyclophosphamide, epirubicin, raloxifene, toremifene, letrozole, trastuzumab, megastrol, tamoxifen, paclitaxel, docetaxel, capecitabine, goserelin acetate, zoledronic acid, fluorouracil, everolimus, bevacizumab, bortezomib, cisplatin, methotrexate, adriamycin, daunomycin, vincristine, vinblastine, vinarelbine, hydroxyurea, or any combination of these.

The compounds disclosed herein may be included in pharmaceutical compositions (including therapeutic and prophylactic formulations), typically combined together with one or more pharmaceutically acceptable vehicles or carriers and, optionally, other therapeutic ingredients (for example, antibiotics, anti-inflammatories, or drugs that are used to reduce pruritus such as an antihistamine). The compositions disclosed herein may be advantageously combined and/or used in combination with other anticancer agents as described above.

Such pharmaceutical compositions can be administered to subjects by a variety of mucosal administration modes, including by oral, rectal, intranasal, intrapulmonary, or transdermal delivery, or by topical delivery to other surfaces. Optionally, the compositions can be administered by non-mucosal routes, including by intramuscular, subcutaneous, intravenous, intra-arterial, intra-articular, intraperitoneal, intrathecal, intracerebroventricular, or parenteral routes. In other alternative embodiments, the compound can be administered ex vivo by direct exposure to cells, tissues or organs originating from a subject.

To formulate the pharmaceutical compositions, the compound can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The compound can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The compound can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In accordance with the various treatment methods of the disclosure, the compound can be delivered to a subject in a manner consistent with conventional methodologies associated with management of the disorder for which treatment or prevention is sought. In accordance with the disclosure herein, a prophylactically or therapeutically effective amount of the compound and/or other biologically active agent is administered to a subject in need of such treatment for a time and under conditions sufficient to prevent, inhibit, and/or ameliorate a selected disease or condition or one or more symptom(s) thereof.

Typical subjects intended for treatment with the compositions and methods of the present disclosure include humans, as well as non-human primates and other animals. To identify subjects for prophylaxis or treatment according to the methods of the disclosure, accepted screening methods are employed to determine risk factors associated with a cancer to determine the status of an existing disease or condition in a subject. For example, a subject that has been found to have the greatest percentage of stem cells in their tumor at the time of initial biopsy may be suitable candidates for treatment with the agents described herein. The administration of the compound of the disclosure can be for either prophylactic or therapeutic purpose. When provided prophylactically, the compound is provided in advance of any symptom. The prophylactic administration of the compound serves to prevent or ameliorate any subsequent disease process. When provided therapeutically, the compound is provided at (or shortly after) the onset of a symptom of disease or infection. For prophylactic and therapeutic purposes, the compound can be administered to the subject by the oral route or in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, avian, porcine, feline, non-human primate, and other accepted animal model subjects known in the art. Alternatively, effective dosages can be determined using in vitro models. In alternative embodiments, an effective amount or effective dose of the compound may simply inhibit or enhance one or more selected biological activities correlated with a disease or condition, as set forth herein, for either therapeutic or diagnostic purposes. The actual dosage of the compound will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the compound for eliciting the desired activity or biological response in the subject. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects. A non-limiting range for a therapeutically effective amount of a compound and/or other biologically active agent within the methods and formulations of the disclosure is about 0.01 mg/kg body weight to about 20 mg/kg body weight, such as about 0.05 mg/kg to about 5 mg/kg body weight, or about 0.2 mg/kg to about 2 mg/kg body weight.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation, for example, of an intrapulmonary spray versus powder, sustained release oral versus injected particulate or transdermal delivery formulations, and so forth.

The instant disclosure also includes kits, packages and multi-container units containing the herein described pharmaceutical compositions, active ingredients, and/or means for administering the same for use in the prevention and treatment of diseases and other conditions in mammalian subjects. Kits for diagnostic use are also provided. In one embodiment, these kits include a container or formulation that contains one or more of the conjugates described herein. In one example, this component is formulated in a pharmaceutical preparation for delivery to a subject. The conjugate is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

### EXAMPLES

Breast cancer cells have a lower cloning efficiency and arise more slowly than TACs. MCF7 breast cancer cells were assessed for CD44 and CD24 expression, which can be used to identify and isolate CSCs. As shown in FIG. 1A, CD44hi/CD24lo cells comprised ca. 1% of the entire population, in keeping with previous observations in both MCF7 cells and primary cancers. These cells were then sorted into 96 well plates and expanded as single cell clones. The remainder of the population, consisting of the bulk of the cells, and comprised almost exclusively of TACs, was similarly sorted into a separate set of 96 well plates. All plates were examined daily and the point at which macroscopically visible colonies first became detectable was noted. As seen in FIG. 1B, the TAC population showed a cloning efficiency of 34%; clones were first detected 10 days after plating, and no additional clones were detected beyond day 16. In contrast, CSCs cloned less efficiently (14%), were not first detected until day 14, and continued to appear until day 20. Very similar results were obtained in two repeat experiments. These findings are consistent with a previous report that MCF7 CSCs express higher levels of genes associated with quiescence. It also suggests that, at least when isolated as single cells, CSCs may initially divide more slowly than TACs, as evidenced by the delayed timing of their clonal expansion. Once reaching this multi-cell stage, however, no significant differences were observed in the growth rates of these two populations.

CD44hi/CD24lo CSCs rapidly differentiate. After expanding clonal populations from the above two groups, cell surface expression of CD44 and CD24 were evaluated. As seen in FIG. 2, CSC clones rapidly reverted to a phenotype closely resembling that of the unsorted MCF7 population. Therefore, as predicted, initially pure CSCs rapidly generate a more differentiated population comprised primarily of TACs or TAC-like cells.

GFP tagging of CSCs. A commonly used maker for CSCs, as well as for embryonal stem cells, is Oct3/4 a member of the POU family of transcription factors. In order to identify

CSCs whose fates could be easily followed, unfractionated MCF7 cells were stably transfected with a plasmid encoding green fluorescent protein (GFP) under the control of a 4 kb segment of the human Oct3/4 proximal promoter. As expected, <1% of the resultant transfectants expressed GFP (FIG. 3A), in keeping with the results shown in FIG. 1A. Experiments were then performed to determine whether there was concordance between GFP expression and the CD44hi/CD24lo breast CSC phenotype. This was done in two ways. First, cell sorting was used to isolate GFP+ cells and then these cells were evaluated for CD44 and CD24 expression. As seen in FIG. 3B, virtually all of the GFP+ cells were CD44hi/CD24lo. The reciprocal experiment was also performed in which CD44hi/CD24lo CSCs were isolated and then assessed for expression of GFP. As seen in FIG. 3C, the entire CSC population also expressed GFP. Second, the morphologies and sizes of the two cell populations were evaluated. As seen in FIG. 4, CD44hi/CD24lo cells were distinctly smaller and rounder than the bulk of the MCF7 population.

Oct3/4-GFP+MCF7 cells remain “frozen” in a stem cell-like state. Because the Oct3/4-GFP+MCF7 population was concordant with the CD44hi/CD24lo CSC population, it had initially been expected that it would eventually differentiate into a TAC or TAC-like population as shown in FIG. 2. Thus, it had been predicted that these cells would not only lose their CD44hi/CD24lo phenotype but their expression of GFP as well due to a silencing of the Oct3/4 promoter as the cells differentiated into TACs. Surprisingly, it was found that both the cell surface phenotype and GFP positivity persisted. In fact, these phenotypes have remained stable after >20 weeks of in vitro passage. Attempts have been made to force these cells to differentiate in vitro by a variety of methods including co-culturing them with MCF7 TACs but have been uniformly unsuccessful.

The persistence of the Oct3/4-GFP+MCF7 (and CD44hi/CD24lo) population suggested that these cells might in fact be true CSCs that for some reason had been “locked” or “frozen” in a CSC-like state. In order to further test this idea and to attempt another means of forcing the cells to differentiate into TACs, the capacity of the cells for de novo tumor initiation was tested. 10⁴ of these cells were inoculated subcutaneously in nude mice; concurrently 5×10⁶ unfractionated MCF7 cells were inoculated in a different group of animals. In the latter case, 3/3 animals developed tumors, which first became apparent 6-8 weeks after inoculation. In contrast, 3/3 animals inoculated with the much smaller number of Oct3/4-GFP+MCF7 cells (10⁴/animal) developed tumors by 2 weeks, consistent with the known high efficiency of CSCs purified by CD44/CD24 selection. Thus, Oct3/4-GFP+MCF7 cells were at least 500-fold better than their TAC counterparts at de novo tumor initiation, which is considered to be the “gold standard” for the CSC.

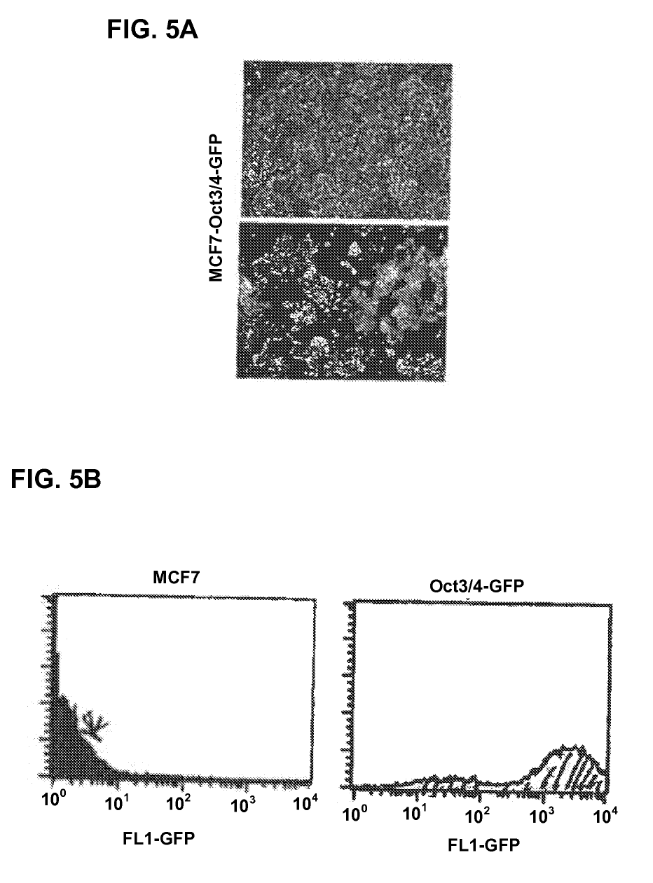

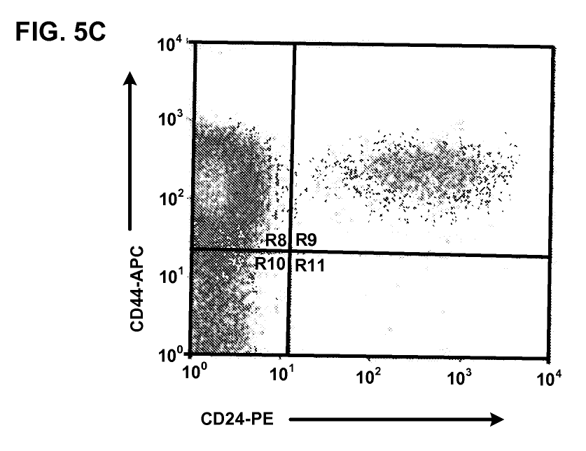

The tumor cell population arising from the initial inoculums of these Oct3/4-GFP+MCF7 cells was further examined. Single cell suspensions were prepared from the tumors, grown for two weeks in G418-containing medium to eliminate host cells, and again analyzed for CSC-like properties. The recovered tumor cells retained full express of both GFP and the CSC phenotype (CD44hi/CD24lo:>95%) (FIG. 5).

In other studies, Oct3/4-GFP+MCF7 cells were examined by qRT-PCR for expression of the breast cancer CSC markers CD133 and Oct3/4. After normalizing to GAPDH, the ratios of these transcripts in Oct3/4-GFP+MCF7 cells and non-CSC-MCF7 cells were 6.3 for CD 133 and 2.5 for Oct3/4. It was also asked whether Oct3/4-GFP+MCF7 cells and non-CSC-MCF7 cells showed differences in the so-called “side population” (SP) in which CSCs have been shown to reside. The SP is due to CSCs ability to exclude the dye Hoechst 33342 and reflects high expression of ABCG2 transporters such as MDR1 and BRCP. The foregoing results showed the SP fraction to comprise 6.3% of MCF7 cells and 23.8% of the Oct3/4-GFP+MCF7cell population. These fractions could also be reduced by >70% following exposure to 20 μM Reserpine, which inhibits ABC transporters. Moreover, the overall mean fluorescence intensity of the entire Oct3/4-GFP+MCF population was two-fold lower than that of the MCF7population. Thus, based on independent qRT-PCR assessments of CSC gene expression profiles and on uptake/exclusion of Hoechst 33342, one can conclude that the Oct3/4-GFP+MCF7 population consists of highly enriched and stable cells that are unable to differentiate into TACs.

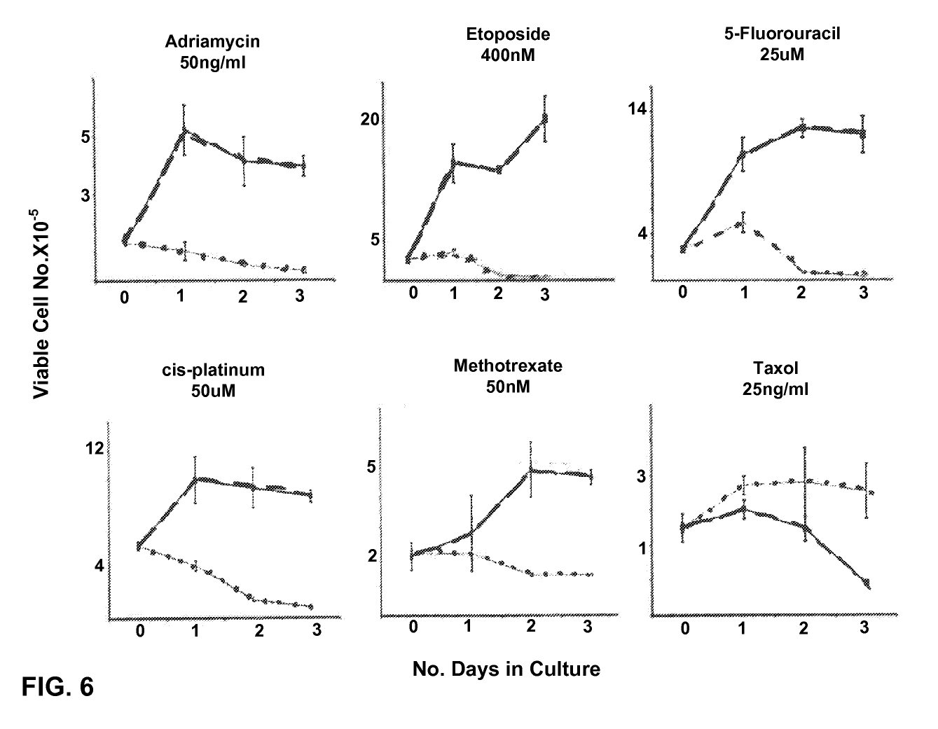

Differential chemosensitivities of Oct3/4-GFP+MCF7 cells. It is believed that CSCs are more resistant to chemotherapeutic agents than TACs, thus at least partly accounting for the propensity of many tumors to relapse after an initial response. To test this idea, MCF7 cells and Oct3/4-GFP+MCF7 CSCs were exposed to six different chemotherapeutic agents commonly employed to treat breast cancer. As seen in FIG. 6, Oct3/4-GFP+MCF7 cells showed significantly greater resistance in five of the six cases and, in fact, even increased in number over the course of the study. The exception to this was seen in the case of taxol where Oct3/4-GFP+MCF cells were significantly more sensitive than MCF7 cells.

A highly sensitive assay for compounds with selectivity for CSCs. The identification of compounds that selectively target Oct3/4-GFP+MCF7 cells demands a screening strategy that is simple, sensitive, rapid, and reproducible. Ideally, it should also be internally controlled to allow for the detection of what might initially be only small differences between the two cell populations. To this end, control MCF7 cells were tagged with the red fluorescent protein DsRED (Clontech). It was shown that the overall fluorescence intensity of this population as well as that of the Oct3/4-GFP+MCF7 CSCs varies in direct proportion to the number of initially plated cells. Further, GFP and DsRED fluorescence were simultaneously measured at non-overlapping emission/excitation wavelengths, and fractional changes of the two populations were reproducibly measured (FIG. 7). Such a labeled population may be used to identify compounds selectively inhibitory to Oct3/4-GFP+MCF7 cells by detecting reductions in the GFP:DsRED ratio. The assay has been adapted from a 96 well plate to a 384 well plate format without loss of sensitivity or specificity.

Selective inhibition of Oct3/4-GFP+MCF cells with small molecules. Oct3/4-GFP+

MCF7 cells were used as the target population. Control cells consisted of Oct3/4-GFP-cells that were infected with a GFP-expressing lentivirus as a way of controlling for GFP expression. Initial screenings were performed in 384 well plates on triplicate samples. A fluorescent plate reader was used to quantify relative changes in GFP levels after a 3 day exposure to a 5 μM concentrations of each compound. After identifying Rottlerin, A77636 and CGP74514A as potentially selective agents, each compound was re-tested at several concentrations for its ability to inhibit each cell line. These experiments were performed in 12 well plates in which 2000 cells/well were initially seeded and allowed to attach for one day. The following day (day 0), the medium was replaced with fresh medium containing the indicated concentrations of each compound. Cell counts were then performed manually on triplicate wells. Bars represent standard errors. The results shown in FIG. 11 were performed with compound concentrations that demonstrated the greatest differences between the two cell populations.

The cell viability assay was performed as follows:

1. Cell preparation:

Trypsinize cell and resuspend in MCF7 culture medium at a density of 4×10⁴/ml Culture medium: alpha MEM, 10% FBS, 2 mM Glutamine, 1 mM Sodium Pyruvate, 1 mM non-essential amino acids, 100 U/ml Penicillin/Streptomycin

2. Screen:

(1) dilute compounds to 30 μM using alpha MEM

(2) add 5 μl compounds to each well (final concentration 5 μM)

(3) add 25 μl cell suspension to each well (1000 cells/well)

(4) incubate cells with compounds for 72 hours at 37° C., 5% CO₂

3. Access cell viability using CellTiter-Blue assay with the following modifications
a. The plate is 384-well plate. There was 30 u1 medium in each well, so 6 u1 CellTiter-Blue was added to each well.
b. 6 u1 of CellTiter-Blue reagent was added to each well and incubated the plates at 37 degree for 2.5 hours. The plates were then read on SpectraMax M5 Microplate Reader using the wavelength of excitation 560 nm/emission 590 nm.

Further illustrative examples are described in the following numbered paragraphs:

1. A method of inhibiting a carcinoma in a subject, comprising administering to the subject at least one therapeutic agent that selectively targets carcinoma stem cells.
2. A method of selectively targeting carcinoma stem cells, comprising administering to a population of carcinoma cells comprising carcinoma stem cells as well as carcinoma cells that are not stem cells at least one anti-cancer agent selected from:

(a) a purine compound, or a pharmaceutically acceptable salt or ester thereof, having a structure represented by

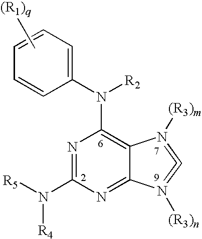

in which q is 1-5,

R₁ is halogen, lower alkyl, hydroxyl or lower alkanoyloxy; lower alkoxy which is unsubstituted or substituted by hydroxyl, lower alkoxy or carboxyl; a radical of the formula —O(—CH₂—CH₂—O)ₜ—R₆, in which t is 2-5 and R₆ is hydrogen or lower alkyl; carboxyl, lower alkoxycarbonyl, piperazin-1-yl-carbonyl or carbamoyl; N-lower alkyl-carbamoyl, which is unsubstituted or substituted by hydroxyl or amino in the lower alkyl moiety; N,N-di-lower alkyl-carbamoyl, cyano, nitro, amino, lower alkanoyl amino, lower alkylamino, N,N-di-lower alkylamino, aminosulfonyl or trifluoromethyl, where, if more than one radical R is present in the molecule, these can be identical to or different from one another,

R₂ is hydrogen, carbamoyl or N-lower alkyl-carbamoyl, m and n are each 0 or 1, where m is 0 if n is 1 and m is 1 if n is 0,

R₃ is lower alkyl or phenyl which are unsubstituted or in each case substituted by hydroxyl, lower alkoxy, amino, lower alkylamino or N,N-di-lower alkyl amino, and

i) R₄ is hydrogen, amino, phenylamino, lower alkylamino, hydroxyl, phenoxy, lower alkoxy, acyl having 1-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms and R₅ is amino, phenylamino, lower alkylamino, hydroxyl, phenoxy, lower alkoxy, acyl having 2-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms, or

ii) R₄ and R₅ together are a substituted or unsubstituted alkylene or alkenylene radical having in each case not more than 15 C atoms, in which 1-3 C atoms can be replaced by oxygen, sulfur or nitrogen, and their salts;

(b) a phenolic compound, or a pharmaceutically acceptable salt or ester thereof, having a structure represented by

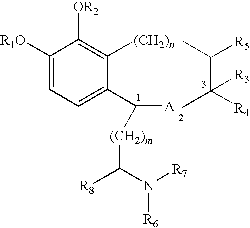

wherein A is O, C, CH, or CH₂;

R₁ and R₂ are independently hydrogen or a leaving group or a protecting group;

m and n are independently selected from zero or 1;

R₃ is H, alkyl, alkenyl, aryl, cycloalkyl, or taken together with R₄ can form a spirocycloalkyl, with the proviso that when n is zero R₃ is not H;

R₄ is H or alkyl, or taken together with R₃ can form a spirocycloalkyl;

R₅ is H or alkyl, or when n is zero, R₅ can be taken together with R₃ to form a fused cycloalkyl;

R₆ is H, alkyl, or taken together with R₈ can form an N containing heterocycle;

R₇ is H, alkyl, alkenyl, cycloalkyl, arylalkyl, or taken together with A when A is C and when m=0 and n=0, can form a fused N containing heterocycle, or taken together with R₈ can form an N containing heterocycle; or R₆ and R₇ together can form an N containing heterocycle with the proviso that when R₆ is alkyl R₇ cannot be arylalkyl;

R₈ is H, alkyl, taken together with R₆ or R₇ to form an N containing heterocycle, or taken together with the catechol ring can form a fused ring; or

(c) a compound, or a pharmaceutically acceptable salt or ester thereof, having a structure represented by

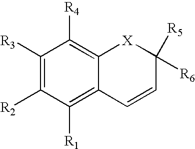

wherein X is selected from the group consisting of CH₂, O, N, and S; R₁ and R₃ are selected from the group consisting of H, OH, NH and SH; R₂ is selected from the group consisting of ethanone, acetyl, alkenyl, aryl and alkyl; R₄ is CO—[CHCH]ₙ-Ph, CN—[CHCH]ₙ-Ph, or COOZ, wherein Z is selected from the group consisting of alkenyl, aryl, and alkyl; R₅ and R₆ are selected from the group consisting of H, OH, NH, SH, alkenyl, aryl, and alkyl; and n is 0 to 5.

3. A method of treating a chemotherapeutic-resistant carcinoma, comprising administering to a subject having a chemotherapeutic-resistant carcinoma at least one therapeutic agent that selectively targets carcinoma stem cells.

4. A method of inhibiting breast cancer in a subject, comprising administering to the subject a therapeutically effective amount of at least one therapeutic agent that selectively targets breast cancer stem cells, the at least one therapeutic agent being selected from:
(a) a purine compound, or a pharmaceutically acceptable salt or ester thereof, having a structure represented by

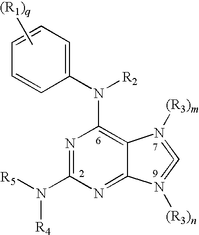

in which q is 1-5,

R₁ is halogen, lower alkyl, hydroxyl or lower alkanoyloxy; lower alkoxy which is unsubstituted or substituted by hydroxyl, lower alkoxy or carboxyl; a radical of the formula —O(—CH₂—CH₂—O)ₜ—R₆, in which t is 2-5 and R₆ is hydrogen or lower alkyl; carboxyl, lower alkoxycarbonyl, piperazin-1-yl-carbonyl or carbamoyl; N-lower alkyl-carbamoyl, which is unsubstituted or substituted by hydroxyl or amino in the lower alkyl moiety; N,N-di-lower alkyl-carbamoyl, cyano, nitro, amino, lower alkanoyl amino, lower alkylamino, N,N-di-lower alkylamino, aminosulfonyl or trifluoromethyl, where, if more than one radical R is present in the molecule, these can be identical to or different from one another,

R₂ is hydrogen, carbamoyl or N-lower alkyl-carbamoyl, m and n are each 0 or 1, where m is 0 if n is 1 and m is 1 if n is 0,

R₃ is lower alkyl or phenyl which are unsubstituted or in each case substituted by hydroxyl, lower alkoxy, amino, lower alkylamino or N,N-di-lower alkyl amino, and

i) R₄ is hydrogen, amino, phenylamino, lower alkylamino, hydroxyl, phenoxy, lower alkoxy, acyl having 1-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms and R₅ is amino, phenylamino, lower alkylamino, hydroxyl, phenoxy, lower alkoxy, acyl having 2-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms, or

ii) R₄ and R₅ together are a substituted or unsubstituted alkylene or alkenylene radical having in each case not more than 15 C atoms, in which 1-3 C atoms can be replaced by oxygen, sulfur or nitrogen, and their salts;

(b) a phenolic compound, or a pharmaceutically acceptable salt or ester thereof, having a structure represented by

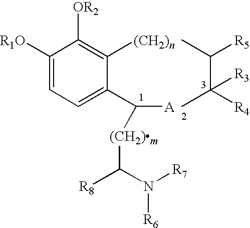

wherein A is O, C, CH, or CH₂;

R₁ and R₂ are independently hydrogen or a leaving group or a protecting group;

m and n are independently selected from zero or 1;

R₃ is H, alkyl, alkenyl, aryl, cycloalkyl, or taken together with R₄ can form a spirocycloalkyl, with the proviso that when n is zero R₃ is not H;

R₄ is H or alkyl, or taken together with R₃ can form a spirocycloalkyl;

R₅ is H or alkyl, or when n is zero, R₅ can be taken together with R₃ to form a fused cycloalkyl;

R₆ is H, alkyl, or taken together with R₈ can form an N containing heterocycle;

R₇ is H, alkyl, alkenyl, cycloalkyl, arylalkyl, or taken together with A when A is C and when m=0 and n=0, can form a fused N containing heterocycle, or taken together with R₈ can form an N containing heterocycle; or R₆ and R₇ together can form an N containing heterocycle with the proviso that when R₆ is alkyl R₇ cannot be arylalkyl;

R₈ is H, alkyl, taken together with R₆ or R₇ to form an N containing heterocycle, or taken together with the catechol ring can form a fused ring; or

(c) a compound, or a pharmaceutically acceptable salt or ester thereof, having a structure represented by

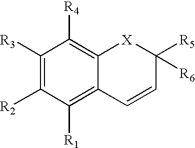

wherein X is selected from the group consisting of CH₂, O, N, and S; R₁ and R₃ are selected from the group consisting of H, OH, NH and SH; R₂ is selected from the group consisting of ethanone, acetyl, alkenyl, aryl and alkyl; R₄ is CO—[CHCH]ₙ-Ph, CN—[CHCH]ₙ-Ph, or COOZ, wherein Z is selected from the group consisting of alkenyl, aryl, and alkyl; R₅ and R₆ are selected from the group consisting of H, OH, NH, SH, alkenyl, aryl, and alkyl; and n is 0 to 5.

5. The method of any one of paragraphs 1 to 3, wherein the carcinoma is a solid tumor.

6. The method of any one of paragraphs 1 to 5, wherein the therapeutic agent or anti-cancer agent is a compound having a molecular weight of less than 600 daltons.

7. The method of any one of paragraphs 1 to 6, wherein the therapeutic agent or anti-cancer agent inhibits proliferation of the stem cells, increases differentiation of the stem cells, induces apoptosis of the stem cells, or a combination thereof.

8. The method of paragraph 1 or 2, wherein the carcinoma is not neuroblastoma.

9. The method of any one of paragraphs 1 to 8, wherein the therapeutic agent or anti-cancer agent is CGP74514A, or a pharmaceutically acceptable salt or ester thereof.

10. The method of any one of paragraphs 1 to 8, wherein the therapeutic agent or anti-cancer agent is rottlerin, or a pharmaceutically acceptable salt or ester thereof.

11. The method of any one of paragraphs 1 to 8, wherein the therapeutic agent or anti-cancer agent is A-77636, or a pharmaceutically acceptable salt or ester thereof.

12. The method of any one of paragraphs 1 to 3 or 5 to 11, wherein the carcinoma is selected from breast cancer, prostate cancer, glioblastoma, colon carcinoma, lung carcinoma, pancreatic cancer, melanoma, gastric cancer, hepatic carcinoma, ovarian carcinoma, or testicular cancer.

13. A pharmaceutical composition useful for selectively targeting carcinoma stem cells, comprising a therapeutically effective amount of at least one therapeutic agent that selectively targets carcinoma stem cells, the at least one therapeutic agent being selected from:
(a) a purine compound, or a pharmaceutically acceptable salt or ester thereof, having a structure represented by

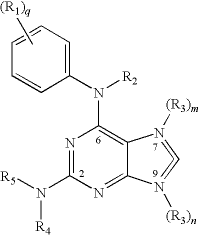

in which q is 1-5,

R₁ is halogen, lower alkyl, hydroxyl or lower alkanoyloxy; lower alkoxy which is unsubstituted or substituted by hydroxyl, lower alkoxy or carboxyl; a radical of the formula —O(—CH₂—CH₂—O)ₜ—R₆, in which t is 2-5 and R₆ is hydrogen or lower alkyl; carboxyl, lower alkoxycarbonyl, piperazin-1-yl-carbonyl or carbamoyl; N-lower alkyl-carbamoyl, which is unsubstituted or substituted by hydroxyl or amino in the lower alkyl moiety; N,N-di-lower alkyl-carbamoyl, cyano, nitro, amino, lower alkanoyl amino, lower alkylamino, N,N-di-lower alkylamino, aminosulfonyl or trifluoromethyl, where, if more than one radical R is present in the molecule, these can be identical to or different from one another,

R₂ is hydrogen, carbamoyl or N-lower alkyl-carbamoyl, m and n are each 0 or 1, where m is 0 if n is 1 and m is 1 if n is 0,

R₃ is lower alkyl or phenyl which are unsubstituted or in each case substituted by hydroxyl, lower alkoxy, amino, lower alkylamino or N,N-di-lower alkyl amino, and

i) R₄ is hydrogen, amino, phenylamino, lower alkylamino, hydroxyl, phenoxy, lower alkoxy, acyl having 1-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms and R₅ is amino, phenylamino, lower alkylamino, hydroxyl, phenoxy, lower alkoxy, acyl having 2-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms, or

ii) R₄ and R₅ together are a substituted or unsubstituted alkylene or alkenylene radical having in each case not more than 15 C atoms, in which 1-3 C atoms can be replaced by oxygen, sulfur or nitrogen, and their salts;

(b) a phenolic compound, or a pharmaceutically acceptable salt or ester thereof, having a structure represented by

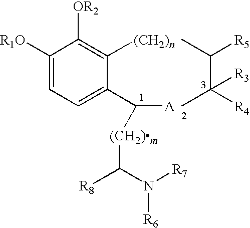

wherein A is O, C, CH, or CH₂;

R₁ and R₂ are independently hydrogen or a leaving group or a protecting group;

m and n are independently selected from zero or 1;

R₃ is H, alkyl, alkenyl, aryl, cycloalkyl, or taken together with R₄ can form a spirocycloalkyl, with the proviso that when n is zero R₃ is not H;

R₄ is H or alkyl, or taken together with R₃ can form a spirocycloalkyl;

R₅ is H or alkyl, or when n is zero, R₅ can be taken together with R₃ to form a fused cycloalkyl;

R₆ is H, alkyl, or taken together with R₈ can form an N containing heterocycle;

R₇ is H, alkyl, alkenyl, cycloalkyl, arylalkyl, or taken together with A when A is C and when m=0 and n=0, can form a fused N containing heterocycle, or taken together with R₈ can form an N containing heterocycle; or R₆ and R₇ together can form an N containing heterocycle with the proviso that when R₆ is alkyl R₇ cannot be arylalkyl;

R₈ is H, alkyl, taken together with R₆ or R₇ to form an N containing heterocycle, or taken together with the catechol ring can form a fused ring; or

(c) a compound, or a pharmaceutically acceptable salt or ester thereof, having a structure represented by

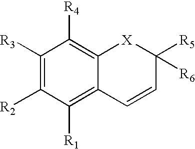

wherein X is selected from the group consisting of CH₂, O, N, and S; R₁ and R₃ are selected from the group consisting of H, OH, NH and SH; R₂ is selected from the group consisting of ethanone, acetyl, alkenyl, aryl and alkyl; R₄ is CO—[CHCH]ₙ-Ph, CN—[CHCH]ₙ-Ph, or COOZ, wherein Z is selected from the group consisting of alkenyl, aryl, and alkyl; R₅ and R₆ are selected from the group consisting of H, OH, NH, SH, alkenyl, aryl, and alkyl; and n is 0 to 5; and

at least one carrier or adjuvant.

In view of the many possible embodiments to which the principles of the disclosed methods may be applied, it should be recognized that the illustrated embodiments are only preferred examples and should not be taken as limiting the scope of the invention.

What is claimed is:

1. A method of inhibiting a carcinoma in a subject, comprising administering to the subject at least one therapeutic agent that selectively targets carcinoma stem cells.

2. A method of selectively targeting carcinoma stem cells, comprising administering to a population of carcinoma cells comprising carcinoma stem cells as well as carcinoma cells that are not stem cells at least one anti-cancer agent selected from:
(a) a purine compound, or a pharmaceutically acceptable salt or ester thereof, having a structure represented by 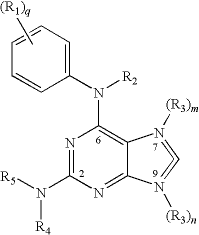
in which q is 1-5,
R₁ is halogen, lower alkyl, hydroxyl or lower alkanoyloxy; lower alkoxy which is unsubstituted or substituted by hydroxyl, lower alkoxy or carboxyl; a radical of the formula —O(—CH₂—CH₂—O)ₜ—R₆, in which t is 2-5 and R₆ is hydrogen or lower alkyl; carboxyl, lower alkoxycarbonyl, piperazin-1-yl-carbonyl or carbamoyl; N-lower alkyl-carbamoyl, which is unsubstituted or substituted by hydroxyl or amino in the lower alkyl moiety; N,N-di-lower alkyl-carbamoyl, cyano, nitro, amino, lower alkanoyl amino, lower alkylamino, N,N-di-lower alkylamino, aminosulfonyl or trifluoromethyl, where, if more than one radical R is present in the molecule, these can be identical to or different from one another,
R₂ is hydrogen, carbamoyl or N-lower alkyl-carbamoyl, m and n are each 0 or 1, where m is 0 if n is 1 and m is 1 if n is O,
R₃ is lower alkyl or phenyl which are unsubstituted or in each case substituted by hydroxyl, lower alkoxy, amino, lower alkylamino or N,N-di-lower alkyl amino, and
i) R₄ is hydrogen, amino, phenylamino, lower alkylamino, hydroxyl, phenoxy, lower alkoxy, acyl having 1-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms and R₅ is amino, phenylamino, lower alkylamino, hydroxyl, phenoxy, lower alkoxy, acyl having 2-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms, or
ii) R₄ and R₅ together are a substituted or unsubstituted alkylene or alkenylene radical having in each case not more than 15 C atoms, in which 1-3 C atoms can be replaced by oxygen, sulfur or nitrogen, and their salts;
(b) a phenolic compound, or a pharmaceutically acceptable salt or ester thereof, having a structure represented by 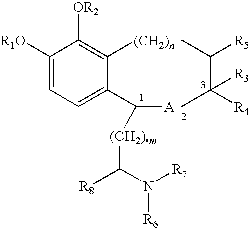
wherein A is O, C, CH, or CH₂;
R₁ and R₂ are independently hydrogen or a leaving group or a protecting group;
m and n are independently selected from zero or 1;
R₃ is H, alkyl, alkenyl, aryl, cycloalkyl, or taken together with R₄ can form a spirocycloalkyl, with the proviso that when n is zero R₃ is not H;
R₄ is H or alkyl, or taken together with R₃ can form a spirocycloalkyl;
R₅ is H or alkyl, or when n is zero, R₅ can be taken together with R₃ to form a fused cycloalkyl;
R₆ is H, alkyl, or taken together with R₈ can form an N containing heterocycle;
R₇ is H, alkyl, alkenyl, cycloalkyl, arylalkyl, or taken together with A when A is C and when m=0 and n=0, can form a fused N containing heterocycle, or taken together with R₈ can form an N containing heterocycle; or R₆ and R₇ together can form an N containing heterocycle with the proviso that when R₆ is alkyl R₇ cannot be arylalkyl;
R₈ is H, alkyl, taken together with R₆ or R₇ to form an N containing heterocycle, or taken together with the catechol ring can form a fused ring; or
(c) a compound, or a pharmaceutically acceptable salt or ester thereof, having a structure represented by 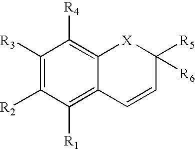
wherein X is selected from the group consisting of CH₂, O, N, and S; R₁ and R₃ are selected from the group consisting of H, OH, NH and SH; R₂ is selected from the group consisting of ethanone, acetyl, alkenyl, aryl and alkyl; R₄ is CO—[CHCH]ₙ-Ph, CN—[CHCH]ₙ-Ph, or COOZ, wherein Z is selected from the group consisting of alkenyl, aryl, and alkyl; R₅ and R₆ are selected from the group consisting of H, OH, NH, SH, alkenyl, aryl, and alkyl; and n is 0 to 5.

3. A method of treating a chemotherapeutic-resistant carcinoma, comprising administering to a subject having a chemotherapeutic-resistant carcinoma at least one therapeutic agent that selectively targets carcinoma stem cells.

4. A method of inhibiting breast cancer in a subject, comprising administering to the subject a therapeutically effective amount of at least one therapeutic agent that selectively targets breast cancer stem cells, the at least one therapeutic agent being selected from:
(a) a purine compound, or a pharmaceutically acceptable salt or ester thereof, having a structure represented by 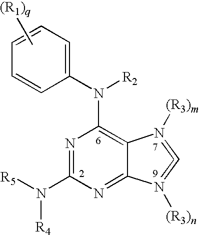
in which q is 1-5,
R₁ is halogen, lower alkyl, hydroxyl or lower alkanoyloxy; lower alkoxy which is unsubstituted or substituted by hydroxyl, lower alkoxy or carboxyl; a radical of the formula —O(—CH₂—CH₂—O)ₜ—R₆, in which t is 2-5 and R₆ is hydrogen or lower alkyl; carboxyl, lower alkoxycarbonyl, piperazin-1-yl-carbonyl or carbamoyl; N-lower alkyl-carbamoyl, which is unsubstituted or substituted by hydroxyl or amino in the lower alkyl moiety; N,N-di-lower alkyl-carbamoyl, cyano, nitro, amino, lower alkanoyl ami no, lower alkylamino, N,N-di-lower alkylamino, aminosulfonyl or trifluoromethyl, where, if more than one radical R is present in the molecule, these can be identical to or different from one another,
R₂ is hydrogen, carbamoyl or N-lower alkyl-carbamoyl, m and n are each 0 or 1, where m is 0 if n is 1 and m is 1 if n is O,
R₃ is lower alkyl or phenyl which are unsubstituted or in each case substituted by hydroxyl, lower alkoxy, amino, lower alkylamino or N,N-di-lower alkyl amino, and
i) R₄ is hydrogen, amino, phenylamino, lower alkylamino, hydroxyl, phenoxy, lower alkoxy, acyl having 1-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms and R₅ is amino, phenylamino, lower alkylamino, hydroxyl, phenoxy, lower alkoxy, acyl having 2-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms, or
ii) R₄ and R₅ together are a substituted or unsubstituted alkylene or alkenylene radical having in each case not more than 15 C atoms, in which 1-3 C atoms can be replaced by oxygen, sulfur or nitrogen, and their salts;
(b) a phenolic compound, or a pharmaceutically acceptable salt or ester thereof, having a structure represented by 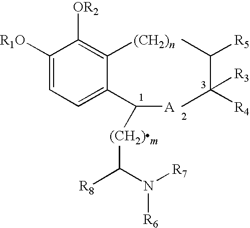
wherein A is O, C, CH, or CH₂;
R₁ and R₂ are independently hydrogen or a leaving group or a protecting group;
m and n are independently selected from zero or 1;
R₃ is H, alkyl, alkenyl, aryl, cycloalkyl, or taken together with R₄ can form a spirocycloalkyl, with the proviso that when n is zero R₃ is not H;
R₄ is H or alkyl, or taken together with R₃ can form a spirocycloalkyl;
R₅ is H or alkyl, or when n is zero, R₅ can be taken together with R₃ to form a fused cycloalkyl;
R₆ is H, alkyl, or taken together with R₈ can form an N containing heterocycle;
R₇ is H, alkyl, alkenyl, cycloalkyl, arylalkyl, or taken together with A when A is C and when m=0 and n=0, can form a fused N containing heterocycle, or taken together with R₈ can form an N containing heterocycle; or R₆ and R₇ together can form an N containing heterocycle with the proviso that when R₆ is alkyl R₇ cannot be arylalkyl;
R₈ is H, alkyl, taken together with R₆ or R₇ to form an N containing heterocycle, or taken together with the catechol ring can form a fused ring; or
(c) a compound, or a pharmaceutically acceptable salt or ester thereof, having a structure represented by 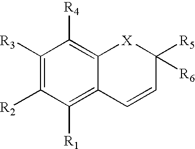
wherein X is selected from the group consisting of CH₂, O, N, and S; R₁ and R₃ are selected from the group consisting of H, OH, NH and SH; R₂ is selected from the group consisting of ethanone, acetyl, alkenyl, aryl and alkyl; R₄ is CO—[CHCH]ₙ-Ph, CN—[CHCH]ₙ-Ph, or COOZ, wherein Z is selected from the group consisting of alkenyl, aryl, and alkyl; R₅ and R₆ are selected from the group consisting of H, OH, NH, SH, alkenyl, aryl, and alkyl; and n is 0 to 5.

5. The method of claim 1, wherein the carcinoma is a solid tumor.

6. The method of claim 2, wherein the carcinoma is a solid tumor.

7. The method of claim 3, wherein the carcinoma is a solid tumor.

8. The method of claim 2, wherein the therapeutic agent or anti-cancer agent is a compound having a molecular weight of less than 600 daltons.

9. The method of claim 2, wherein the therapeutic agent or anti-cancer agent inhibits proliferation of the stem cells, increases differentiation of the stem cells, induces apoptosis of the stem cells, or a combination thereof.

10. The method of claim 2, wherein the carcinoma is not neuroblastoma.

11. The method of claim 2, wherein the therapeutic agent or anti-cancer agent is CGP74514A, or a pharmaceutically acceptable salt or ester thereof.

12. The method of claim 3, wherein the therapeutic agent or anti-cancer agent is CGP74514A, or a pharmaceutically acceptable salt or ester thereof.

13. The method of claim 4, wherein the therapeutic agent or anti-cancer agent is CGP74514A, or a pharmaceutically acceptable salt or ester thereof.

14. The method of claim 2, wherein the therapeutic agent or anti-cancer agent is rottlerin, or a pharmaceutically acceptable salt or ester thereof.

15. The method of claim 3, wherein the therapeutic agent or anti-cancer agent is rottlerin, or a pharmaceutically acceptable salt or ester thereof.

16. The method of claim 4, wherein the therapeutic agent or anti-cancer agent is rottlerin, or a pharmaceutically acceptable salt or ester thereof.

17. The method of claim 2, wherein the therapeutic agent or anti-cancer agent is A-77636, or a pharmaceutically acceptable salt or ester thereof.

18. The method of claim 3, wherein the therapeutic agent or anti-cancer agent is A-77636, or a pharmaceutically acceptable salt or ester thereof.

19. The method of claim 4, wherein the therapeutic agent or anti-cancer agent is A-77636, or a pharmaceutically acceptable salt or ester thereof.

20. The method of claim 2, wherein the carcinoma is selected from breast cancer, prostate cancer, glioblastoma, colon carcinoma, lung carcinoma, pancreatic cancer, melanoma, gastric cancer, hepatic carcinoma, ovarian carcinoma, or testicular cancer.

21. A pharmaceutical composition useful for selectively targeting carcinoma stem cells, comprising a therapeutically effective amount of at least one therapeutic agent that selectively targets carcinoma stem cells, the at least one therapeutic agent being selected from:
(a) a purine compound, or a pharmaceutically acceptable salt or ester thereof, having a structure represented by 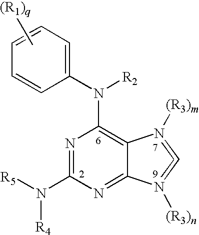
in which q is 1-5,
R₁ is halogen, lower alkyl, hydroxyl or lower alkanoyloxy; lower alkoxy which is unsubstituted or substituted by hydroxyl, lower alkoxy or carboxyl; a radical of the formula —O(—CH₂—CH₂—O)ₜ—R₆, in which t is 2-5 and R₆ is hydrogen or lower alkyl; carboxyl, lower alkoxycarbonyl, piperazin-1-yl-carbonyl or carbamoyl; N-lower alkyl-carbamoyl, which is unsubstituted or substituted by hydroxyl or amino in the lower alkyl moiety; N,N-di-lower alkyl-carbamoyl, cyano, nitro, amino, lower alkanoyl amino, lower alkylamino, N,N-di-lower alkylamino, aminosulfonyl or trifluoromethyl, where, if more than one radical R is present in the molecule, these can be identical to or different from one another,
R₂ is hydrogen, carbamoyl or N-lower alkyl-carbamoyl, m and n are each 0 or 1, where m is 0 if n is 1 and m is 1 if n is 0,
R₃ is lower alkyl or phenyl which are unsubstituted or in each case substituted by hydroxyl, lower alkoxy, amino, lower alkylamino or N,N-di-lower alkyl amino, and
i) R₄ is hydrogen, amino, phenylamino, lower alkylamino, hydroxyl, phenoxy, lower alkoxy, acyl having 1-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms and R₅ is amino, phenylamino, lower alkylamino, hydroxyl, phenoxy, lower alkoxy, acyl having 2-30 C atoms, a substituted aliphatic hydrocarbon radical having not more than 29 C atoms, a carbocyclic radical having not more than 29 C atoms or a heterocyclic radical having not more than 20 C atoms and not more than 9 heteroatoms, or
ii) R₄ and R₅ together are a substituted or unsubstituted alkylene or alkenylene radical having in each case not more than 15 C atoms, in which 1-3 C atoms can be replaced by oxygen, sulfur or nitrogen, and their salts;
(b) a phenolic compound, or a pharmaceutically acceptable salt or ester thereof, having a structure represented by 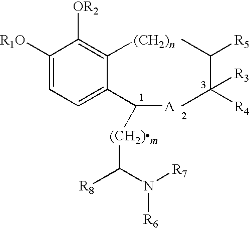
wherein A is O, C, CH, or CH₂;
R₁ and R₂ are independently hydrogen or a leaving group or a protecting group;
m and n are independently selected from zero or 1;
R₃ is H, alkyl, alkenyl, aryl, cycloalkyl, or taken together with R₄ can form a spirocycloalkyl, with the proviso that when n is zero R₃ is not H;
R₄ is H or alkyl, or taken together with R₃ can form a spirocycloalkyl;
R₅ is H or alkyl, or when n is zero, R₅ can be taken together with R₃ to form a fused cycloalkyl;
R₆ is H, alkyl, or taken together with R₈ can form an N containing heterocycle;
R₇ is H, alkyl, alkenyl, cycloalkyl, arylalkyl, or taken together with A when A is C and when m=0 and n=0, can form a fused N containing heterocycle, or taken together with R₈ can form an N containing heterocycle; or R₆ and R₇ together can form an N containing heterocycle with the proviso that when R₆ is alkyl R₇ cannot be arylalkyl;
R₈ is H, alkyl, taken together with R₆ or R₇ to form an N containing heterocycle, or taken together with the catechol ring can form a fused ring; or
(c) a compound, or a pharmaceutically acceptable salt or ester thereof, having a structure represented by 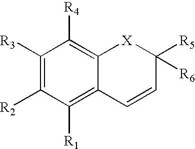
wherein X is selected from the group consisting of CH₂, O, N, and S; R₁ and R₃ are selected from the group consisting of H, OH, NH and SH; R₂ is selected from the group consisting of ethanone, acetyl, alkenyl, aryl and alkyl; R₄ is CO—[CHCH]ₙ-Ph, CN—[CHCH]ₙ-Ph, or COOZ, wherein Z is selected from the group consisting of alkenyl, aryl, and alkyl; R₅ and R₆ are selected from the group consisting of H, OH, NH, SH, alkenyl, aryl, and alkyl; and n is 0 to 5; and
at least one carrier or adjuvant.


SEQUENCE LISTING (8 sequences)

SEQ ID NO: 1 - DNA, 3990 bp, Homo sapiens
tttcctacccccatttctcttcacaggtgtgcagagaagtctacattcccatgtcaagtaagtggggtgggtagatgggagggcatatgagagcggggcagcaattttatgtcttagttggaaggaagtttagagttcattgagtccaaatcctctttactaggtgaggaaactgaggcacaggagggggaggtgccattagccagggagtcagagccaggacttagaacctggtccatgcctcctagtttgttcctgaccaatcctcccagctcacccactccttttgctctcacctctgactccctacaggctacagctagagagttgactcctctatttgaggtaagagtgtctggatttgggataaggtgacctcagtttgcaggtctcatgggggagatcagagggtcctttgttgagaagaggggacttggtccacagtgagcaaaggaatttgtctaaattgctattaagggagatcatctcccaaggtgggccaagcctgaaccctcagccctccatttcttccctttcttttgccttcagattgacaggcctcggaagtcaaaataagtggtttcctagaccgggtcgagagcaagtctctattggtcccaactgagttttttcagctggtttttcaaccaaacagcacctcatctcccagtgaggggaagggaaggctgggctgagagcagcaaggctgctcatctcacctctccccacccagccatgccagccgccacacctggtggggagaggtgggcctcacctgggtcccctggcagtgctctgtgaagggtcttgacattgcactgtaataataaaggtgtgtgtgaagtatctttttatggtgactttctaaaacccagggaatcatgggaccagttctgatgactcagcctggcttccagtctcctccaggcccaacggtggcccccagcactggttggggcctgggagagctggccttggctgaagtgaagccacctacccttcaggcataacaggacagtgagaaggaaggaaagcctgcctcaacctcccatcagccctgagcaccccagaagggggccggctaggagtctaggcatgcaggaggctgacccctgactgggctcatatccagccacaaggcagccagggacccaggcacccaccccttgtctgcgtccctctcgggaatgggcctcttgcccaggccagaaatacaccacctacagtacaaattataatctaaaaacaagagggtggtgttgagtggggaaattggggaaggtgttttaggagccactaggaaaatgggcagcagggactctctggactggcttgggaagagcgcttttggggaacctggaggatggcaagctgagaaacactggtgtggagattccagccaaatcccaggcctgcccctccccctcctctgagaggccgtcttcttggcagacagcagagagatgcatgacaaaggtgccgtgatggttctgtcctggggattgagatggctggggaggggcctcctcctgttccgaagcatgttcctcccacccccaccaggccccataatctacctgccttttgggcagttaaaggccgagaagtgaacacagctgcaaccccactgccttgtagaccttccggcagacctgtggcaggtattgaaatgcacgcatacaattaggctcaaaaagtctacacagacaggagatgggcacacgaacagaggcaacataagagtgggggaaaagtctcaaaagactcacggatgccaccaagatgaagacagctggccacgggacacccatccccttagaaggcagacagagccactgaccccagcagacaagcccaggcagggctgagcctggagcctgcaatgagaagccttacttaagtcgacagaggtcagcgtgcccagtccagacctggccttctggccttcgaagctgtggggagccctggcccagagccccctctggagcccccagacttaccccaggccctccactgagatcaagttttgggagcagacagacaaacatcatccctcacagacaggcattccgttggctattctcttgcaaacagaatcaagcactagaccagcagcatgagcctcaggatactcaggccaggcccagaaaaacagaccctgaaggggagcttagggcagccttcctgcacccctccacaaatcactcgccacctcctctgcgtctttctgccagccagccccactaaacaaagcacatccctcaatcttccgggctcggggagggatgcacgatgaagctggacgcctgagtcccccagaggaaggaggaactagatacctaggtccctgtagggggcccttggtgcccgtctgaggctcagtctttgaggggattgcagaggggggttgctggagctccttttagcgtctctgaaggggattctgtgtgaggggattgggactggggggttggggagcaggaagcagtccccaggggagccatccaggcccattcaagggttgagcacttgtttagggttagagctgccccctctggggaccgggattgtccagccaaggccattgtcctgcccccttcccccagtccctcccaggcttctttgaacctgaagtcagatattttttctccacaccccccaccccctggttttccccacccagggcctagggctggaggcctgggccagggaggtgggggagggagaacggggcctaccgtggtattagatgtctgagttttggttgagaggggagcaaggaacctgatgtgcaggttccatagtggagggggcccaaagcgggtgtcttatcactctgtttcagcaaaggttgggaaactgaggcccaatcagtccaaagtctggtcccttgaaggggaagtaggggccaaccccttagtctgttagatgaggagagtctggagtctgattctggaagacggaggggtggggggatggggggtggggggatatagcacggaggccttgtctggcagtctactcttgaagatggggtgaaatttggcaggctgggcagatggtgccaggcacccaggctgcggggtggctggatttggccagtatcgggatgggaatgcctaggattctggatggatcgggggaaggcataagggagcagctggccattgtgcttatggctgttgatgcattgagggatagcgccacacacacattcaataaatttgaggagctgagagggtgactggcccctgaaggcacagtgccagaggtctgtggagagggggtcaagcacctgggttcctgaagaacatggaggtgtgggagtgattccagacagctgggatgtgcagagcctgagagagtgccagggagcgggttgggagttgaaagttgggtgtggtggctcacgcctttaatcatgacactgggcggcagaggcgggaggatttcttgaggacaggaattcaagaccagcctgggtaacatagcaaggccccatctctactaaaaataaaaaaactaacagggcacagtggtccaagcctgtagtcccagccacttaggaggctggagcagaaggattgctttggcccagtagatcgaggctacattgagccatcattgtactccactgcactccagtctgggcaacaaagtgagaccctgtcttaaaaaataaaaataaaaaaagtttctgtgggggacctgcactgaggtcctggaggggcgccagttgtgtctcccggttttccccttccacagacaccattgccaccaccattaggcaaacatccttcgcctcagtttctccccccacctccctctcctccacccatccagggggcggggccagaggtcaaggctagtgggtgggactggggagggagagaggggttgagtagtcccttcgcaagccctcatttcaccaggcccccggcttggggcgccttccttccccatggcgggacacctggcttcagattttgc

SEQ ID NO: 2 - DNA, 22 bp, Artificial Sequence
  Synthetic Oligonucleotide Primer for ORF138
tggaacctgcacatcaggttcc

SEQ ID NO: 3 - DNA, 21 bp, Artificial Sequence
  Synthetic Oligonucleotide Primer for ORF138 (REV)
tcagtctttgaggggattgca

SEQ ID NO: 4 - DNA, 23 bp, Artificial Sequence
  Synthetic Oligonucleotide Primer for ORF201
tcccagctgtctggaatcactcc

SEQ ID NO: 5 - DNA, 22 bp, Artificial Sequence
  Synthetic Oligonucleotide Primer for ORF201 (REV)
ctaccgtggtattagatgtctg

SEQ ID NO: 6 - DNA, 20 bp, Artificial Sequence
  Synthetic Oligonucleotide Primer for ORF162
tagattatggggcctggtgg

SEQ ID NO: 7 - DNA, 21 bp, Artificial Sequence
  Synthetic Oligonucleotide Primer for ORF162 (REV)
ctaggagtctaggcatgcagg

SEQ ID NO: 8 - DNA, 1890 bp, Murine
aagctttgtgaacttggcggcttccaagtcgctgcctttatttaggtcttccaactaactatggcactgttccacaatgaatgtatagaaattgggaggtgagcatgacagagtggaggaaacggaagattcatggagagggccagagagatggcccctcagccaccctgggggatgacttggacccatgtggtagaaggaggggactttcacacatgtgctatgtgtagctgtgtgtaggtacatacacacccttaaaataaaacgcaatttttttttcaaagtctcagggtgaatttggtgaagtcgatgaagctgaggcaggagaattatcaggagttcaagggcagcttgttttatagagaaaggttccatctctacctgatgaagactaccatcaagagacacccccgccccccagggcacctagagccactgaccctagccaacagctcaggcgggctgggcccaggctcagaactctgtcctggctatgtacactgtggggtgctctgggctttttgaggctgtgtgattcaccctggggccttcgttcagggcatggtgtaggagcagacagacaaacaccatcccttgcagacaggcactctgagggctattctcttgcaaagataactaagcaccaggccagtaatgggatcctcagactgggcccagaaaaccactctaggaaagttcagggtaggctctctgcaccccctcctcctaatcccgtctccttagtgtctttccgccagcacaggaatgggggaggggtgggtgacgaggatgaacaccggagtccctggaggaagggaagcagggtatctccatctgaggctctgtctttgaggagaggtggagagctggggaagtcttgtgtgaggggattggggctcaggagggggttggggagcaggaagttgtccccaggggagccatcctggcccattcaagggttgagtacttgtttagggttagagctgcccctctggggaccaggattgtccagccaaggccattgtcctgcccccttcccccagtccctcccaggcccctttgaacctgaagtcagatatttcttctctctacccacctcccacccgttgggtttctccacccaggaactaggctggaagctgggatgaggaggtgggggagggagaactgagaatcttgaggaaagaggccccggccttaactgtgaggggatggagcctgggtgcaggtcttatgggggttgggggtggttagtgtctaatctaccaacctgacaacacaagatggatactgtgctctgaaaacgcagagccagcacttctctggggtctctggggacatatctggttggggctcggggtcccatggtgtagagcctctaaactctggaggactggaggtgcaatggctgtcttgtcctggccttggacatgggctgaaatactgggttcacccatatctaggactctagacgggtgggtaagcaagaactgaggagtggccccagaaataattggcacacgaacattcaatggatgttttaggctctccagaggatggctgagtgggctgtaaggacaggccgagagggtgcagtgccaacaggctttgtggtgcgatggggcatccgagcaactggtttgtgaggtgtccggtgacccaaggcaggggtgagaggaccttgaaggttgaaaatgaaggccttcctggggtcccgtcctaagggttgtcctgtccagacgtccccaacctccgtctggaagacacaggcagatagcgctcgcctcagtttctccacccccacagctctgctcctccacccacccagggggcggggccagaggtcaaggctagagggtgggattggggagggagaggtgaaaccgtccc